(19) United States
(12) Patent Application Publication
(10) Pub. No.: US 2026/0285891 A1
(43) Pub. Date: Sep. 24, 2026

(54) CYCLOPENTADIENYL DERIVATIVES AND THEIR USES IN CATALYSIS

(71) Applicant: Sigma-Aldrich Co. LLC, St. Louis, MO (US)

(72) Inventors: Tharique Nalakath, Milwaukee, WI (US); Guolin Xu, Menomonee Falls, WI (US); Thomas Colacot, Cherry Hill, NJ (US)

(21) Appl. No.: 19/472,534

(22) PCT Filed: Mar. 21, 2024
(86) PCT No.: PCT/US2024/020960

§ 371 Date: Oct. 3, 2025

Related U.S. Application Data
(60) Provisional application No. 63/494,077, filed on Apr. 4, 2023.

(51) Int. Cl. C07F 17/02

(52) U.S. Cl. CPC C07F 17/02

(57) ABSTRACT

Provided are ferrocenyl-based phosphine compounds, which contain either (i) a di(adamantyl)phosphino group on a first cyclopentadienide ring and a penta-aryl substitution on a second cyclopentadienide ring, or (ii) a di(adamantyl)phosphino group on a first cyclopentadienide ring and a second cyclopentadienide ring, respectively. Further provided are scalable processes for synthesizing said ferrocenyl-based phosphine compounds starting from readily available starting materials with simple workup as well as various precatalysts containing a transition metal and said ferrocenyl-based phosphine compounds as ligands.

The present application claims the benefit of priority of U.S. provisional patent application No. 63/494,077, filed on Apr. 4, 2023, the content of which is hereby incorporated in its entirety.

### FIELD OF INVENTION

Provided are new ferrocenyl-based electron-rich phosphine compounds, which contain either (i) a di(adamantyl)phosphino group on a first cyclopentadienide ring and a penta-aryl substitution on a second cyclopentadienide ring (AdQPhos type), or (ii) a di(adamantyl)phosphino group on a first cyclopentadienide ring and a second cyclopentadienide ring, respectively (AdMPhos type).

Further provided are scalable processes for synthesizing said new ferrocenyl-based electron-rich phosphine compounds starting from readily available starting materials with simple workup.

Further provided are various precatalysts containing a transition metal and said new ferrocenyl-based electron-rich phosphine compounds as ligands. Such precatalysts include, for example, (a) Ar—X or X—X transition metal complexes of the type LMArX (Formula VI) and LMX₂ (Formula VII), (b) R-allyl transition metal complexes of the type LM(R-allyl) X (Formula VIII) and LM(R-allyl) (Formula IX), and (c) N-biphenyl transition metal complexes of the type LM(biphenyl-NR) X (Formula X) and LM(biphenyl-NR)⁺ (Formula XI) as shown in FIG. 1, which are useful in catalysis.

All compounds and complexes prepared herein were thoroughly characterized by various analytical techniques such as NMR, elemental analyses, single crystal X-ray, etc., Superior catalytic activities in many types of cross-coupling reactions such as, for example (1) C(sp²)-C(sp²) coupling reactions, (2) C(sp²)-C(sp³) coupling reactions, (3) C(sp²)-N coupling reactions, (4) C(sp²)-O coupling reactions, (5) C(sp²)-S coupling reactions, (6) C(sp²)-P coupling reactions or (7) α-arylations of amides, esters, nitriles, nitroalkanes, ketones, etc., were demonstrated in comparison with QPhos and other ligands known in the state of the art.

Hence, there are further provided methods for performing transition metal-catalyzed coupling reactions between a first substrate and a second substrate, wherein said transition metal-catalyzed coupling reactions are preferably selected from C(sp²)-C(sp²) coupling reactions, C(sp²)-C(sp³) coupling reactions, C(sp²)-N coupling reactions, C(sp²)-O coupling reactions, C(sp²)-S coupling reactions, C(sp²)-P coupling reactions or α-arylations of amides, esters, nitriles, nitroalkanes or ketones. FIG. 2 shows non-limiting examples of the reactions mentioned.

### BACKGROUND

The rise of palladium as the metal of the 21^{st} century is due to its applications in catalysis,⁽¹⁾ where the role of phosphine ligands is paramount. These ligands by virtue of their electronic, steric, and other kinetic/thermodynamic factors define their overall efficacy and efficiency in catalysis. Ferrocene-based bis phosphines constitute an important class of modern ligands, which are extensively used in various cross-coupling reactions, such as, for example, dtbpf, dppf, dippf, MPhos.⁽²⁾

The synthesis and application studies associated with ferrocenyl monophosphines were limited due to various challenges associated with their synthesis, purification and scale-up. Sollot and co-workers reported the first monosubstituted ferrocenyl phosphine via Friedel-Craft's route in 1962 (suffered poor yield and selectivity);⁽³⁾ later, pioneering works by Knox & Pauson,⁽⁴⁾ Juge & Genet,⁽⁵⁾ and Jamison⁽⁶⁾ outlined general strategies for installing dialkyl/aryl phosphine motifs into the ferrocene ring.

Despite these advances, the direct application of these ligands in cross-coupling reactions remained limited until early 2000. Hartwig and co-workers reported the synthesis of pentaphenyl ferrocenyl phosphines (also known as QPhos) and their derivatives in 2002.⁽⁷⁾ QPhos was relatively stable compared to other non-arylated species and exhibited unconventional catalytic activity towards various C—C, C—O, and C—N cross-coupling reactions, although the scale up of this ligand was quite challenging due to the involvement of chromatography. The scalability challenges of this exceptionally good ligand compared to the corresponding bisphosphinyl ferrocenes limited its industrial applications.⁽⁸⁾ The precatalysts derived from these new ligands exhibit superior activity compared to other commercially available ligands including QPhos.⁽⁹⁾

Despite these advances, a need still exists for new ligands that (1) can address some of the existing challenges in cross coupling, and (2) which have syntheses that are amenable to scale up in manufacture sufficient to prepare sufficient quantities at a purity acceptable for industrial applications.

In view of this, the present invention addresses two major advancements in this field: (1) the development of a new class of electron-rich polyarylated ferrocenyl monophosphines and their transition metal complexes, and (2) the process development/optimization for their scalable syntheses for their use in commercial applications. This process does not rely on any exhaustive purification techniques such as chromatography.

In summary, the present invention relates to the synthesis and catalytic application of a new class of air-stable polyarylated ferrocenyl monophosphines and their transition metal complexes, along with a green route for their scalable synthesis.

### SUMMARY

Provided are new ferrocenyl-based electron-rich phosphine compounds, which contain either (i) a di(adamantyl)phosphino group on a first cyclopentadienide ring and a penta-aryl substitution on a second cyclopentadienide ring (AdQPhos type), or (ii) a di(adamantyl)phosphino group on a first cyclopentadienide ring and a second cyclopentadienide ring, respectively (AdMPhos type).

Hence, in a first embodiment of the present invention, a compound of Formula I is provided:

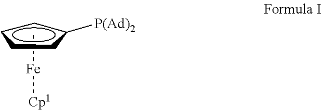

wherein:
   Cp¹ is represented by Formula II or Formula III

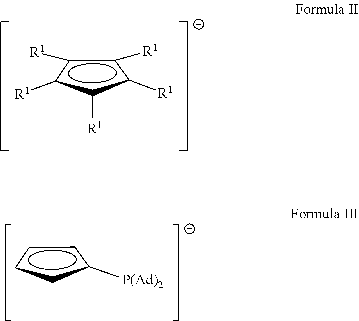

R¹ is at each occurrence independently from each other selected from optionally substituted C₆-C₁₀ aryl or C₃-C₉ heteroaryl;
Ad is adamantyl, preferably 1-adamantyl; and
each optional substituent, when present, is selected from the group consisting of C₁-C₄ alkyl, C₁-C₄ haloalkyl, and C₁-C₄ alkoxy, preferably methyl, trifluoromethyl, and methoxy.

In a second embodiment of the present invention, a process for synthesizing a compound according to Formula IV is provided, wherein the process comprises the following steps:
(a) lithiation of a compound according to Formula A and reacting it with Ad₂PY² to obtain a compound according to Formula B; and
(b) reacting the compound according to Formula B with R¹Y³ in the presence of a base and a catalytic amount of Pd(OAc)₂ to obtain a compound according to Formula IV;

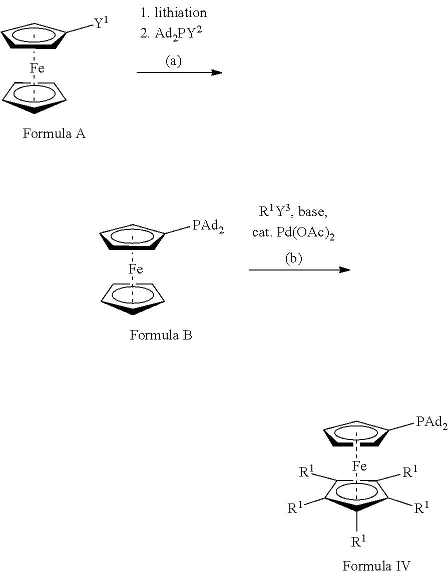

wherein:
   Y¹ is selected from a halogen atom or a hydrogen atom;
   Y², Y³ are independently from each other selected from a halogen atom;
   R¹ is at each occurrence independently from each other selected from optionally substituted C₆-C₁₀ aryl or C₃-C₉ heteroaryl;
   Ad is adamantyl, preferably 1-adamantyl; and
   each optional substituent, when present, is selected from the group consisting of C₁-C₄ alkyl, C₁-C₄ haloalkyl, and C₁-C₄ alkoxy, preferably methyl, trifluoromethyl, and methoxy.

In a third embodiment of the present invention, a process for synthesizing a compound according to Formula V is provided, wherein the process comprises the following step:
(a′) lithiation of a compound according to Formula C and reacting it with Ad₂PY² to obtain a compound according to Formula V:

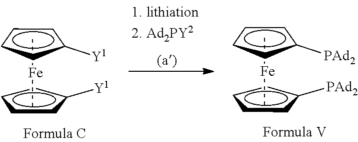

wherein:
   Y¹ is selected from a halogen atom or a hydrogen atom;
   Y² are independently from each other selected from a halogen atom; and
   Ad is adamantyl, preferably 1-adamantyl.

In a fourth embodiment of the present invention, a precatalyst of Formula VI or Formula VII is provided:

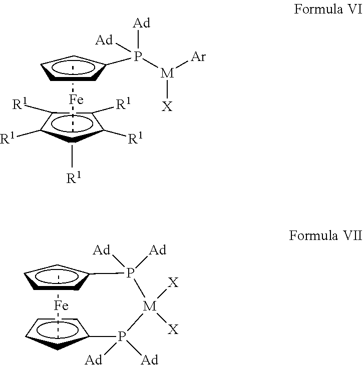

wherein:
   R¹ is at each occurrence independently from each other selected from optionally substituted C₆-C₁₀ aryl or C₃-C₉ heteroaryl;
   Ad is adamantyl, preferably 1-adamantyl;
   M is a transition metal selected from Group 9, Group 10 or Group 11;
   X is selected from the group consisting of halide, triflate (TfO⁻), tetrafluoroborate (BF₄⁻), hexafluorophosphate (PF₆⁻), mesylate (MsO⁻), tosylate (TsO⁻), tetrakis[3,5-bis(trifluoromethyl)phenyl]borate (B[(CF₃)₂C₆H₃]₄⁻), hexafluoro-antimonate (SbF₆⁻) and combinations thereof;
   Ar is optionally substituted C₆-C₁₀ aryl; and
   each optional substituent, when present, is selected from the group consisting of C₁-C₄ alkyl, C₁-C₄ haloalkyl, and C₁-C₄ alkoxy, preferably methyl, trifluoromethyl, and methoxy.

In a fifth embodiment of the present invention, a precatalyst of Formula VIII or Formula IX is provided:

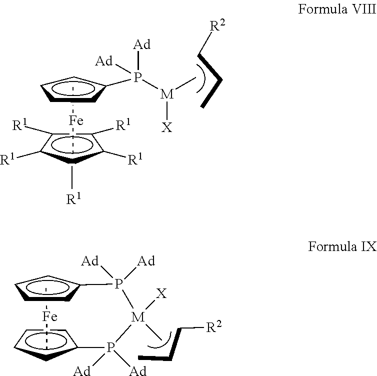

wherein:
   R¹ is at each occurrence independently from each other selected from optionally substituted C₆-C₁₀ aryl or C₃-C₉ heteroaryl;
   Ad is adamantyl, preferably 1-adamantyl;
   M is a transition metal selected from Group 9, Group 10 or Group 11;
   X is selected from the group consisting of halide, triflate (TfO⁻), tetrafluoroborate (BF₄⁻), hexafluorophosphate (PF₆⁻), mesylate (MsO⁻), tosylate (TsO⁻), tetrakis[3,5-bis(trifluoromethyl)phenyl]borate (B[(CF₃)₂C₆H₃]₄⁻), hexafluoro-antimonate (SbF₆⁻) and combinations thereof;
   R² is selected from the group consisting of H, C₁-C₄ alkyl and C₆-C₁₀ aryl, preferably selected from the group consisting of H, Me and Ph; and
   each optional substituent, when present, is selected from the group consisting of C₁-C₄ alkyl, C₁-C₄ haloalkyl, and C₁-C₄ alkoxy, preferably methyl, trifluoromethyl, and methoxy.

In a sixth embodiment of the present invention, a precatalyst of Formula X or Formula XI is provided:

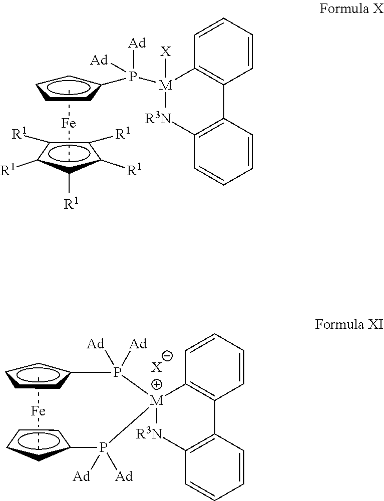

wherein:
   R¹ is at each occurrence independently from each other selected from optionally substituted C₆-C₁₀ aryl or C₃-C₉ heteroaryl;
   Ad is adamantyl, preferably 1-adamantyl;
   M is a transition metal selected from Group 9, Group 10 or Group 11;
   X is selected from the group consisting of halide, triflate (TfO⁻), tetrafluoroborate (BF₄⁻), hexafluorophosphate (PF₆⁻), mesylate (MsO⁻), tosylate (TsO⁻), tetrakis[3,5-bis(trifluoromethyl)phenyl]borate (B[(CF₃)₂C₆H₃]₄⁻), hexafluoro-antimonate (SbF₆⁻) and combinations thereof;
   R³ is selected from the group consisting of H, Me, NHMe, Ph, and NHPh; and
   each optional substituent, when present, is selected from the group consisting of C₁-C₄ alkyl, C₁-C₄ haloalkyl, and C₁-C₄ alkoxy, preferably methyl, trifluoromethyl, and methoxy.

In a seventh embodiment of the present invention, a method for performing a transition metal-catalyzed coupling reaction between a first substrate and a second substrate, wherein the method comprises the following steps:
(a) providing (i) a compound according to the first embodiment of the present invention and a transition metal source, or (ii) a precatalyst according to any one of the fourth, fifth or sixth embodiment of the present invention in a reaction vessel;
(b) adding a first substrate and a second substrate to the reaction vessel; and
(c) reacting the first substrate and the second substrate at a temperature and a time sufficient to perform a transition metal-catalyzed coupling reaction.

### BRIEF DESCRIPTION OF THE FIGURES

FIG. 1 shows various precatalysts: (a) Ar—X or X—X transition metal complexes of the type LMArX (Formula VI) and LMX₂ (Formula VII), (b) R-allyl transition metal complexes of the type LM(R-allyl) X (Formula VIII) and LM(R-allyl) (Formula IX), and (c) N-biphenyl transition metal complexes of the type LM(biphenyl-NR) X (Formula X) and LM(biphenyl-NR)′ (Formula XI).

FIG. 2 shows examples of the following transition metal-catalyzed coupling reactions: a) C(sp²)-C(sp²) coupling reaction, b) C(sp²)-C(sp³) coupling reaction, c) C(sp²)-N coupling reaction, d) C(sp²)-O coupling reaction, e) C(sp²)-S coupling reaction, f) α-arylation of ketones, g) α-arylation of nitriles, h) α-arylation of esters, i) α-arylation of nitroalkanes, j) α-arylation of amides, and k) C(sp²)-P coupling reactions, wherein when R is attached to a heteroatom (e.g. N, P, etc.), R denotes hydrogen or a substituent, and wherein when R is attached to an aromatic ring system, R denotes hydrogen or one or more substituents.

### DETAILED DESCRIPTION

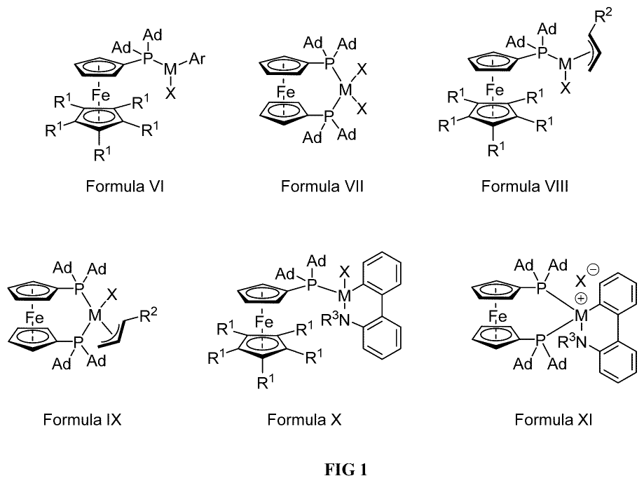

The new compounds and precatalysts described herein, such as those shown in FIG. 1, overcome the problems with conventional catalysts and provide powerful new routes to previously challenging cross-coupling reactions, while being scalable so that they can be obtained and provided in sufficient quantity and purity for industrial applications.

These new compounds and precatalysts are based on a ferrocenyl backbone and contain either (i) a di(adamantyl)phosphino group on a first cyclopentadienide ring and a penta-aryl substitution on a second cyclopentadienide ring, or (ii) a di(adamantyl)phosphino group on a first cyclopentadienide ring and a second cyclopentadienide ring, respectively. As described herein, they provide significant advantages over existing ligands and precatalysts.

Conventional processes for synthesizing ferrocenyl-based phosphine compounds are not suitable to incorporate two Ad₂P moieties into a ferrocenyl compound to prepare compounds of the type Fc (Ad₂P)(Ad₂P).

Herein described are new ferrocenyl-based electron-rich phosphine compounds containing either (i) a di(adamantyl)phosphino group on a first cyclopentadienide ring and a penta-aryl substitution on a second cyclopentadienide ring (AdQPhos type), or (ii) a di(adamantyl)phosphino group on a first cyclopentadienide ring and a second cyclopentadienide ring, respectively (AdMPhos type).

Further provided are scalable syntheses of said new ferrocenyl-based electron-rich phosphine compounds starting from readily available starting materials with simple workup.

Further provided are precatalysts containing a transition metal and said new ferrocenyl-based electron-rich phosphine compounds as ligands as shown in FIG. 1, which are useful in catalysis.

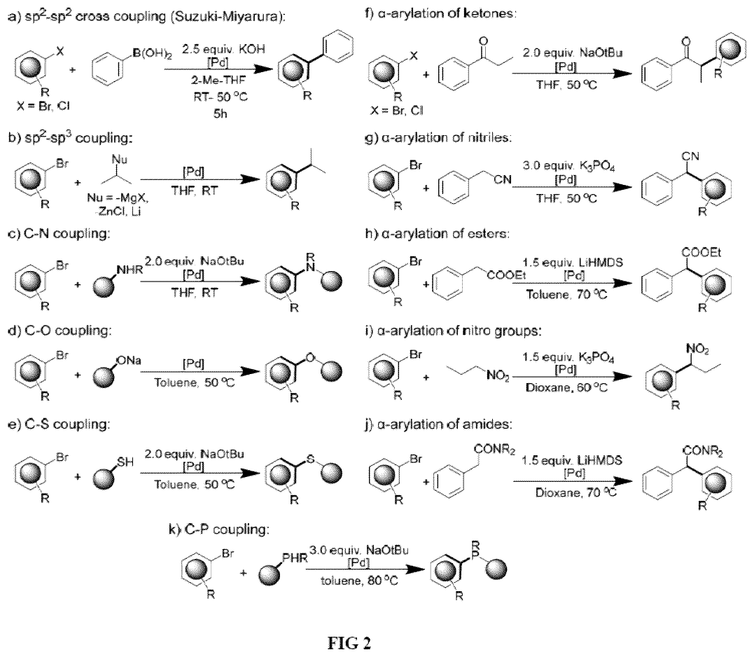

Also provided are methods for performing transition metal-catalyzed coupling reactions between a first substrate and a second substrate, wherein said transition metal-catalyzed coupling reactions are preferably selected from C(sp²)-C(sp²) coupling reactions, C(sp²)-C(sp³) coupling reactions, C(sp²)-N coupling reactions, C(sp²)-O coupling reactions, C(sp²)-S coupling reactions, C(sp²)-P coupling reactions or α-arylations of amides, esters, nitriles, nitroalkanes or ketones. See FIG. 2 for non-limiting examples of the reactions mentioned.

In a first embodiment of the present invention, a compound of Formula I is provided:

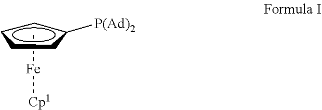

wherein:
   Cp¹ is represented by Formula II or Formula III

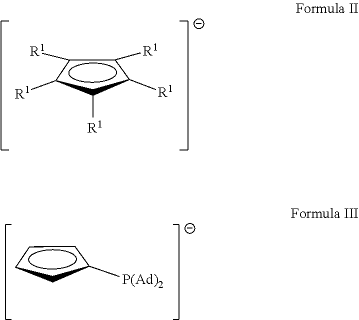

R¹ is at each occurrence independently from each other selected from optionally substituted C₆-C₁₀ aryl or C₃-C₉ heteroaryl;
Ad is adamantyl, preferably 1-adamantyl; and
each optional substituent, when present, is selected from the group consisting of C₁-C₄ alkyl, C₁-C₄ haloalkyl, and C₁-C₄ alkoxy, preferably methyl, trifluoromethyl, and methoxy.

In a preferred embodiment of the present invention, the compound of Formula I is represented by Formula IV:

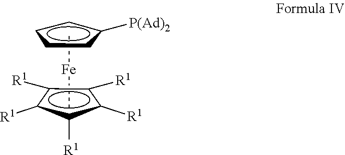

wherein:
   R¹ is at each occurrence independently from each other selected from optionally substituted C₆-C₁₀ aryl or C₃-C₉ heteroaryl; and
   Ad is adamantyl, preferably 1-adamantyl.

Preferably, in Formula II and/or Formula IV of the present invention, R¹ is at each occurrence independently from each other selected from optionally substituted C₆ aryl; and each optional substituent, when present, is selected from the group consisting of C₁-C₄ alkyl, C₁-C₄ haloalkyl, and C₁-C₄ alkoxy, preferably methyl, trifluoromethyl, and methoxy. More preferably, in Formula II and/or Formula IV of the present invention, R¹ is at each occurrence independently from each other selected from the group consisting of phenyl, tolyl, and (trifluoromethyl)phenyl. Most preferably, in Formula II and/or Formula IV of the present invention, R¹ is at each occurrence independently from each other selected from the group consisting of phenyl, p-tolyl, and p-(trifluoromethyl)phenyl.

In a preferred embodiment of the present invention, the compound of Formula I is represented by Formula V:

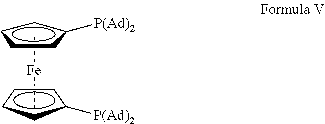

wherein Ad is adamantyl, preferably 1-adamantyl.

In a more preferred embodiment of the present invention, the compound of Formula I is selected from the group consisting of

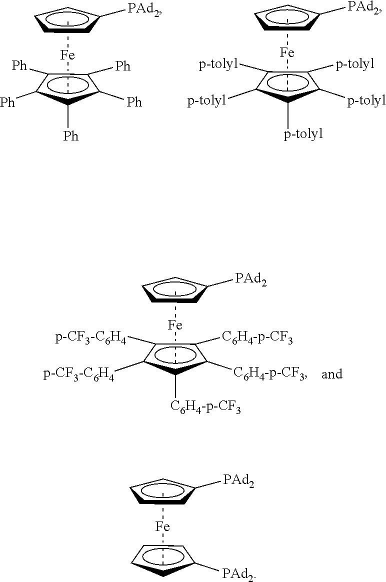

In a second embodiment of the present invention, a process for synthesizing a compound according to Formula IV is provided, wherein the process comprises the following steps:
(a) lithiation of a compound according to Formula A and reacting it with Ad₂PY² to obtain a compound according to Formula B; and
(b) reacting the compound according to Formula B with R¹Y³ in the presence of a base and a catalytic amount of Pd(OAc)₂ to obtain a compound according to Formula IV;

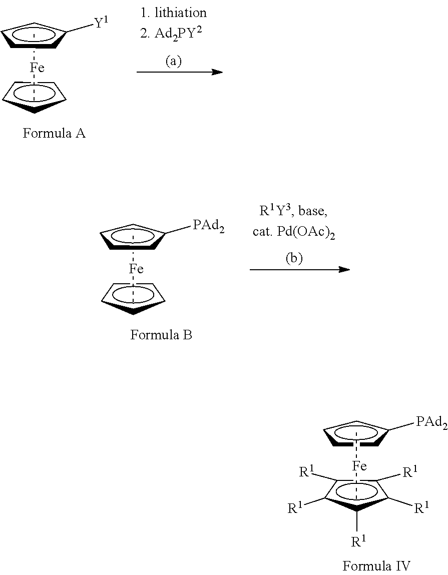

wherein:
   Y¹ is selected from a halogen atom or a hydrogen atom;
   Y², Y³ are independently from each other selected from a halogen atom;
   R¹ is at each occurrence independently from each other selected from optionally substituted C₆-C₁₀ aryl or C₃-C₉ heteroaryl;
   Ad is adamantyl, preferably 1-adamantyl; and
   each optional substituent, when present, is selected from the group consisting of C₁-C₄ alkyl, C₁-C₄ haloalkyl, and C₁-C₄ alkoxy, preferably methyl, trifluoromethyl, and methoxy.

Preferably, Y¹ is selected from the group consisting of Cl, Br, I and H; and Y², Y³ are independently from each other selected from the group consisting of Cl, Br, and I. More preferably, Y¹ is Br or H; Y² is Cl; and Y³ is Cl.

Preferred, more preferred and most preferred embodiments for R¹ in the second embodiment are the same as given above for the first embodiment.

Preferably, the lithiation in step (a) is carried out by reacting the compound according to Formula A with an organolithium reagent. More preferably, the lithiation in step (a) is carried out by reacting the compound according to Formula A with an organolithium reagent selected from the group consisting of nBuLi, sBuLi, and tBuLi. Most preferably, the lithiation in step (a) is carried out by reacting the compound according to Formula A with nBuLi.

Preferably, step (a) is carried out in an ether solvent. More preferably, step (a) is carried out in tetrahydrofuran.

Preferably, the lithiation in step (a) is carried out at a temperature of about −78° C. More preferably, the lithiation in step (a) is carried out at a temperature of about −78° C. and the reaction with Ad₂PY² in step (a) is carried out at a temperature gradient from about −78° C. to room temperature.

Preferably, the base in step (b) is an alkali metal alkoxide or an alkaline earth metal alkoxide. More preferably, the base in step (b) is a sodium alkoxide. Most preferably, the base in step (b) is NaOtBu.

Preferably, step (b) is carried out at a temperature in the range from about 80° C. to about 130° C. More preferably, step (b) is carried out at a temperature in the range from about 100° C. to about 120° C. Most preferably, step (b) is carried out at a temperature of about 110° C.

In a third embodiment of the present invention, a process for synthesizing a compound according to Formula V is provided, wherein the process comprises the following step:
(a′) lithiation of a compound according to Formula C and reacting it with Ad₂PY² to obtain a compound according to Formula V:

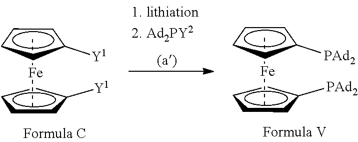

wherein:
   Y¹ is selected from a halogen atom or a hydrogen atom;
   Y² is selected from a halogen atom; and
   Ad is adamantyl, preferably 1-adamantyl.

Preferably, Y¹ is selected from the group consisting of Cl, Br, I and H; and Y² is selected from the group consisting of Cl, Br, and I. More preferably, Y¹ is Br or H; and Y² is Cl.

Preferably, the lithiation in step (a′) is carried out by reacting the compound according to Formula C with an organolithium reagent. More preferably, the lithiation in step (a′) is carried out by reacting the compound according to Formula C with an organolithium reagent selected from the group consisting of nBuLi, sBuLi, and tBuLi. Most preferably, the lithiation in step (a′) is carried out by reacting the compound according to Formula C with nBuLi.

Preferably, step (a′) is carried out in an ether solvent. More preferably, step (a′) is carried out in tetrahydrofuran.

Preferably, the lithiation in step (a′) is carried out at a temperature of about −78° C. More preferably, the lithiation in step (a′) is carried out at a temperature of about −78° C. and the reaction with Ad₂PY² in step (a′) is carried out at a temperature gradient from about −78° C. to room temperature.

In a fourth embodiment of the present invention, a precatalyst of Formula VI or Formula VII is provided:

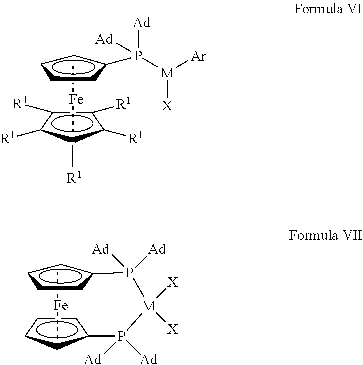

wherein:
   R¹ is at each occurrence independently from each other selected from optionally substituted C₆-C₁₀ aryl or C₃-C₉ heteroaryl;
   Ad is adamantyl, preferably 1-adamantyl;
   M is a transition metal selected from Group 9, Group 10 or Group 11;
   X is selected from the group consisting of halide, triflate (TfO⁻), tetrafluoroborate (BF₄⁻), hexafluorophosphate (PF₆⁻), mesylate (MsO⁻), tosylate (TsO⁻), tetrakis└3,5-bis(trifluoromethyl)phenyl┘borate (B└(CF₃)₂C₆H₃┘₄⁻), hexafluoro-antimonate (SbF₆⁻) and combinations thereof;
   Ar is optionally substituted C₆-C₁₀ aryl; and
   each optional substituent, when present, is selected from the group consisting of C₁-C₄ alkyl, C₁-C₄ haloalkyl, and C₁-C₄ alkoxy, preferably methyl, trifluoromethyl, and methoxy.

Preferably, in Formula VI of the present invention, R¹ is at each occurrence independently from each other selected from optionally substituted C₆ aryl; and each optional substituent, when present, is selected from the group consisting of C₁-C₄ alkyl, C₁-C₄ haloalkyl, and C₁-C₄ alkoxy, preferably methyl, trifluoromethyl, and methoxy. More preferably, in Formula VI of the present invention, R¹ is at each occurrence independently from each other selected from the group consisting of phenyl, tolyl, and (trifluoromethyl)phenyl. Most preferably, in Formula VI of the present invention, R¹ is at each occurrence independently from each other selected from the group consisting of phenyl, p-tolyl, and p-(trifluoromethyl)phenyl.

Preferably, in Formula VI and/or Formula VII of the present invention, M is selected from the group consisting of Co, Rh, Ir, Ni, Pd, Pt, Cu, Ag, and Au. More preferably, in Formula VI and/or Formula VII of the present invention, M is selected from the group consisting of Ni, Pd, and Pt. Most preferably, in Formula VI and/or Formula VII of the present invention, Mis Pd.

Preferably, in Formula VI and/or Formula VII of the present invention, X is selected from the group consisting of chloride (Cl⁻), bromide (Br⁻), iodide (I⁻), triflate (TfO⁻), mesylate (MsO⁻), tosylate (TsO⁻) and combinations thereof.

Preferably, in Formula VI of the present invention, Ar is optionally substituted C₆ aryl. More preferably, in Formula VI of the present invention, Ar is selected from the group consisting of Ph, tolyl, and (trifluoromethyl)phenyl. Most preferably, in Formula VI of the present invention, Ar is selected from the group consisting of p-tolyl, and p-(trifluoromethyl)phenyl.

In a preferred embodiment of the present invention, the precatalyst of Formula VI or Formula VII is selected from the group consisting of

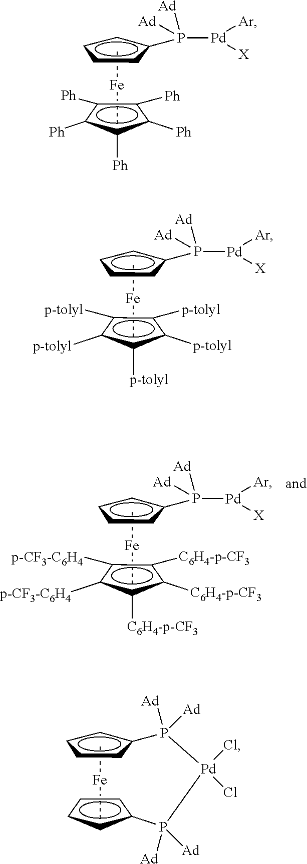

wherein:
   Ad is 1-adamantyl;
   Ar is Ph or p-(trifluoromethyl)phenyl; and
   X is selected from the group consisting of chloride (Cl⁻), bromide (Br⁻), triflate (TfO⁻) and mesylate (MsO⁻).

In a fifth embodiment of the present invention, a precatalyst of Formula VIII or Formula IX is provided:

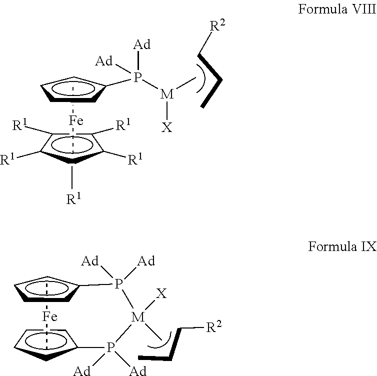

wherein:
   R¹ is at each occurrence independently from each other selected from optionally substituted C₆-C₁₀ aryl or C₃-C₉ heteroaryl;
   Ad is adamantyl, preferably 1-adamantyl;
   M is a transition metal selected from Group 9, Group 10 or Group 11;
   X is selected from the group consisting of halide, triflate (TfO⁻), tetrafluoroborate (BF₄⁻), hexafluorophosphate (PF₆⁻), mesylate (MsO⁻), tosylate (TsO⁻), tetrakis[3,5-bis(trifluoromethyl)phenyl]borate (B[(CF₃)₂C₆H₃]₄⁻), hexafluoro-antimonate (SbF₆⁻) and combinations thereof;
   R² is selected from the group consisting of H, C₁-C₄ alkyl and C₆-C₁₀ aryl, preferably selected from the group consisting of H, Me and Ph; and
   each optional substituent, when present, is selected from the group consisting of C₁-C₄ alkyl, C₁-C₄ haloalkyl, and C₁-C₄ alkoxy, preferably methyl, trifluoromethyl, and methoxy.

Preferably, in Formula VIII of the present invention, R¹ is at each occurrence independently from each other selected from optionally substituted C₆ aryl; and each optional substituent, when present, is selected from the group consisting of C₁-C₄ alkyl, C₁-C₄ haloalkyl, and C₁-C₄ alkoxy, preferably methyl, trifluoromethyl, and methoxy. More preferably, in Formula VIII of the present invention, R¹ is at each occurrence independently from each other selected from the group consisting of phenyl, tolyl, and (trifluoromethyl)phenyl. Most preferably, in Formula VIII of the present invention, R¹ is at each occurrence independently from each other selected from the group consisting of phenyl, p-tolyl, and p-(trifluoromethyl)phenyl.

Preferably, in Formula VIII and/or Formula IX of the present invention, Mis selected from the group consisting of Co, Rh, Ir, Ni, Pd, Pt, Cu, Ag, and Au. More preferably, in Formula VIII and/or Formula IX of the present invention, M is selected from the group consisting of Ni, Pd, and Pt. Most preferably, in Formula VIII and/or Formula IX of the present invention, M is Pd.

Preferably, in Formula VIII and/or Formula IX of the present invention, X is selected from the group consisting of chloride (Cl⁻), bromide (Br⁻), iodide (I⁻), triflate (TfO⁻), tetrafluoroborate (BF₄⁻), hexafluorophosphate (PF₆⁻), mesylate (MsO⁻), tosylate (TsO⁻) and combinations thereof.

Preferably, in Formula VIII and/or Formula IX of the present invention, R² is selected from the group consisting of H, Me and Ph.

In a preferred embodiment of the present invention, the precatalyst of Formula VIII or Formula IX is selected from the group consisting of

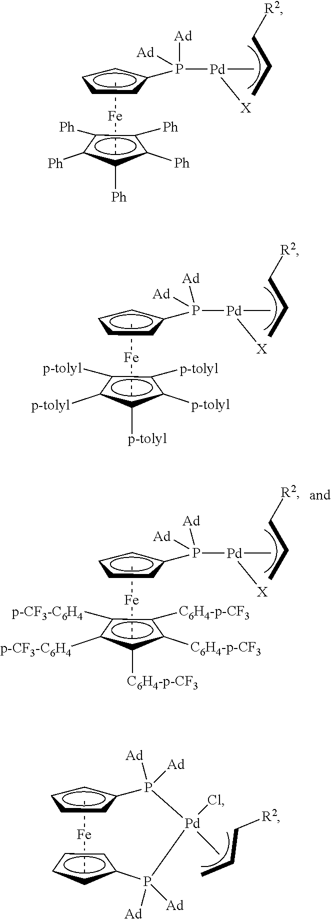

wherein:
   Ad is 1-adamantyl;
   R² is selected from the group consisting of H, Me, and Ph; and
   X is selected from the group consisting of chloride (Cl⁻), bromide (Br⁻), iodide (I⁻), triflate (TfO⁻), tetrafluoroborate (BF₄⁻) and hexafluorophosphate (PF₆⁻).

In a sixth embodiment of the present invention, a precatalyst of Formula X or Formula XI is provided:

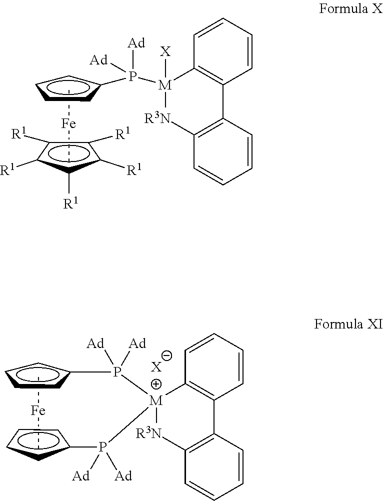

wherein:
   R¹ is at each occurrence independently from each other selected from optionally substituted C₆-C₁₀ aryl or C₃-C₉ heteroaryl;
   Ad is adamantyl, preferably 1-adamantyl;
   M is a transition metal selected from Group 9, Group 10 or Group 11;
   is selected from the group consisting of halide, triflate (TfO⁻), tetrafluoroborate (BF₄⁻), hexafluorophosphate (PF₆⁻), mesylate (MsO⁻), tosylate (TsO⁻), tetrakis[3,5-bis(trifluoromethyl)phenyl]borate (B[(CF₃)₂C₆H₃]₄⁻), hexafluoro-antimonate (SbF₆⁻) and combinations thereof;
   R³ is selected from the group consisting of H, Me, NHMe, Ph, and NHPh; and
   each optional substituent, when present, is selected from the group consisting of C₁-C₄ alkyl, C₁-C₄ haloalkyl, and C₁-C₄ alkoxy, preferably methyl, trifluoromethyl, and methoxy.

Preferably, in Formula X of the present invention, R¹ is at each occurrence independently from each other selected from optionally substituted C₆ aryl; and each optional substituent, when present, is selected from the group consisting of C₁-C₄ alkyl, C₁-C₄ haloalkyl, and C₁-C₄ alkoxy, preferably methyl, trifluoromethyl, and methoxy. More preferably, in Formula X of the present invention, R¹ is at each occurrence independently from each other selected from the group consisting of phenyl, tolyl, and (trifluoromethyl)phenyl. Most preferably, in Formula X of the present invention, R¹ is at each occurrence independently from each other selected from the group consisting of phenyl, p-tolyl, and p-(trifluoromethyl)phenyl.

Preferably, in Formula X and/or Formula XI of the present invention, M is selected from the group consisting of Co, Rh, Ir, Ni, Pd, Pt, Cu, Ag, and Au. More preferably, in Formula X and/or Formula XI of the present invention, M is selected from the group consisting of Ni, Pd, and Pt. Most preferably, in Formula X and/or Formula XI of the present invention, M is Pd.

Preferably, in Formula X and/or Formula XI of the present invention, X is selected from the group consisting of chloride (Cl⁻), bromide (Br⁻), iodide (I⁻), triflate (TfO⁻), tetrafluoroborate (BF₄⁻), hexafluorophosphate (PF₆⁻), mesylate (MsO⁻), tosylate (TsO⁻) and combinations thereof.

Preferably, in Formula X and/or Formula XI of the present invention, R³ is selected from the group consisting of H, Me and NHMe.

In a preferred embodiment of the present invention, the precatalyst of Formula X or Formula XI is selected from the group consisting of

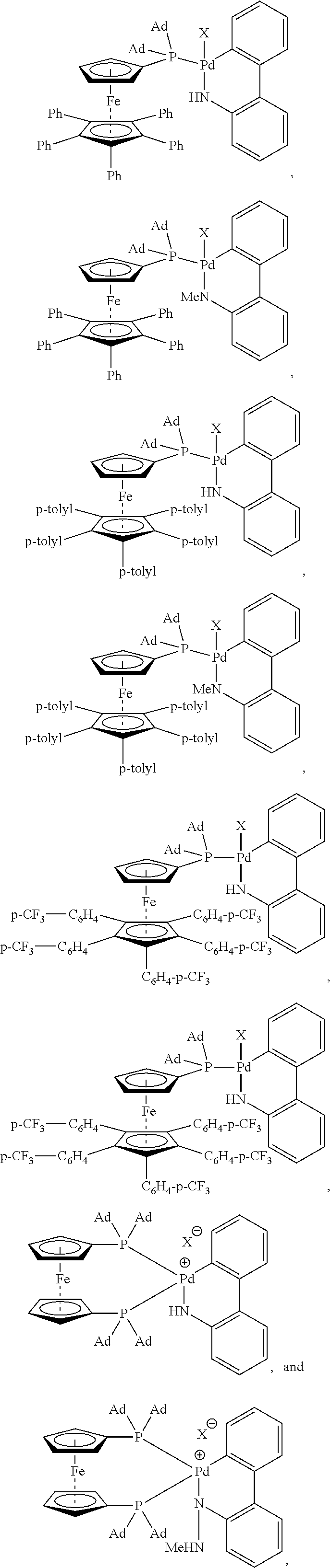

wherein:
   Ad is 1-adamantyl; and
   X is mesylate (MsO⁻).

In a seventh embodiment of the present invention, a method for performing a transition metal-catalyzed coupling reaction between a first substrate and a second substrate is provided, wherein the method comprises the following steps:
(a) providing (i) a compound according to the first embodiment of the present invention and a transition metal source, or (ii) a precatalyst according to any one of the fourth, fifth or sixth embodiment of the present invention in a reaction vessel;
(b) adding a first substrate and a second substrate to the reaction vessel; and
(c) reacting the first substrate and the second substrate at a temperature and a time sufficient to perform a transition metal-catalyzed coupling reaction.

In a preferred embodiment of the present invention, the transition metal source in the seventh embodiment is a Pd metal source and the precatalyst is a Pd precatalyst.

In a more preferred embodiment of the present invention, the transition metal source in the seventh embodiment is selected from the group consisting of Pd(cod)X₂, [Pd(allyl)X]₂, [Pd(crotyl)X]₂, [Pd(cinnamyl)X]₂, [(2-biphenyl-NHR) Pd(OMs)]₂, (cod)Pd(CH₂CMe₂C₆H₄), (cod)Pd(CH₂TMS)₂, Pd(dba)₂, Pd₂(dba)₃, and PdX₂(CH₃CN)₂; wherein cod=1,5-cyclooctadiene; X=Cl or Br; R=H, Me or Ph; Ms=methylsulfonyl; TMS=trimethylsilyl; and dba=dibenzylideneacetone.

In a most preferred embodiment of the present invention, the transition metal source in the seventh embodiment is selected from the group consisting of Pd(cod)Cl₂, [Pd(allyl)Cl]₂, [Pd(crotyl)Cl]₂, [Pd(cinnamyl)Cl]₂, [(2-biphenyl-NHR)Pd(OMs)]₂, (cod)Pd(CH₂CMe₂C₆H₄), (cod)Pd(CH₂TMS)₂, Pd(dba)₂, Pd₂(dba)₃, and PdCl₂(CH₃CN)₂; wherein cod=1,5-cyclooctadiene; R=H, Me or Ph; Ms=methylsulfonyl; TMS=trimethylsilyl; and dba=dibenzylideneacetone.

In a preferred embodiment of the present invention, the transition metal-catalyzed coupling reaction in the seventh embodiment is selected from a C(sp²)-C(sp²) coupling reaction, a C(sp²)-C(sp³) coupling reaction, a C(sp²)-N coupling reaction, a C(sp²)-O coupling reaction, a C(sp²)-S coupling reaction, a C(sp²)-P coupling reaction or an α-arylation of an amide, ester, nitrile, nitroalkane or ketone.

Preferably, the temperature in step (c) of the method for performing a transition metal-catalyzed coupling reaction according to the present invention is in the range from room temperature to 130° C., more preferably from room temperature to 100° C., and most preferably from room temperature to 70° C.

In a preferred embodiment of the present invention, the compound provided in step (a) of the method for performing a transition metal-catalyzed coupling reaction according to the present invention is selected from the group consisting of:

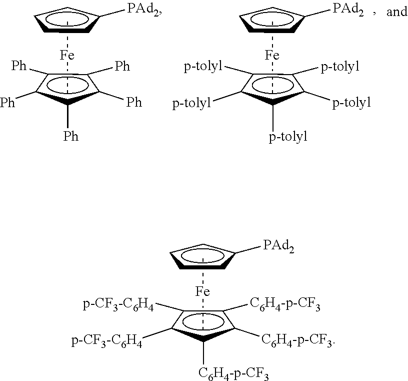

In a preferred embodiment of the present invention, the precatalyst provided in step (a) of the method for performing a transition metal-catalyzed coupling reaction according to the present invention is selected from the group consisting of:

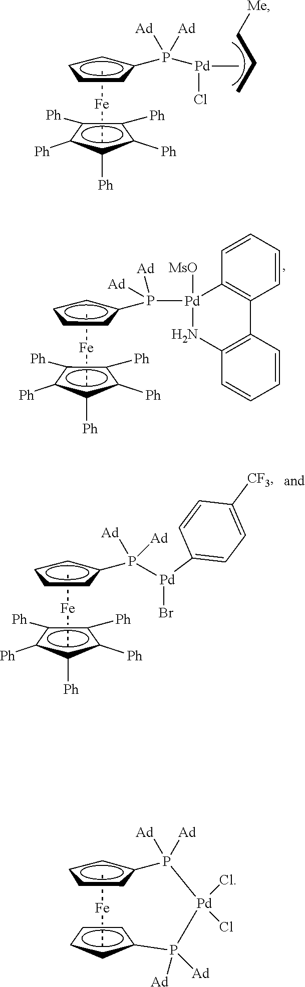

In a preferred embodiment of the present invention, the first substrate provided in step (b) of the method for performing a transition metal-catalyzed coupling reaction according to the present invention is selected from an aromatic compound, which is substituted with a halogen, preferably Br or Cl, and optionally contains one or more substituents selected from the group consisting of alkyl, alkoxy, aryl, aryloxy, heteroaryl, heteroaryloxy, halogen, hydroxy, nitro, and nitrile.

In a more preferred embodiment of the present invention, the first substrate provided in step (b) of the method for performing a transition metal-catalyzed coupling reaction according to the present invention is selected from a C₆-C₁₈ aromatic compound, which is substituted with a halogen, preferably Br or Cl, and optionally contains one or more substituents selected from the group consisting of C₁-C₁₀ alkyl, C₁-C₁₀ alkoxy, C₆-C₁₀ aryl, C₆-C₁₀ aryloxy, C₃-C₉ heteroaryl. C₃-C₉ heteroaryloxy, F, Br, Cl, I hydroxy, nitro, and nitrile.

In a most preferred embodiment of the present invention, the first substrate provided in step (b) of the method for performing a transition metal-catalyzed coupling reaction according to the present invention is selected from a Co aromatic compound, which is substituted with Br or Cl, and optionally contains one or more substituents selected from the group consisting of C₁-C₅alkyl, C₁-C₅alkoxy, C₆ aryl, C₆ aryloxy, F, Br, Cl, I hydroxy, nitro, and nitrile.

In a preferred embodiment of the present invention, the second substrate provided in step (b) of the method for performing a transition metal-catalyzed coupling reaction according to the present invention is selected from the group consisting of alkyl metal halides, alkoxides, boronic acids, thiols, ketones, amides, nitroalkanes, nitriles, esters, and phosphines.

In a more preferred embodiment of the present invention, the second substrate provided in step (b) of the method for performing a transition metal-catalyzed coupling reaction according to the present invention is selected from the group consisting of alkyl magnesium halides containing 1 to 20 carbon atoms, alkyl zinc halides containing 1 to 20 carbon atoms, alkoxides containing 1 to 20 carbon atoms, boronic acids containing 1 to 20 carbon atoms, alkyl thiols containing 1 to 20 carbon atoms, ketones containing 2 to 20 carbon atoms, amides containing 2 to 20 carbon atoms, nitroalkanes containing 1 to 20 carbon atoms, nitriles containing 1 to 20 carbon atoms, esters containing 2 to 20 carbon atoms, and phosphines containing 1 to 20 carbon atoms, which are optionally substituted with one or more substituents selected from the group consisting of fluoro, chloro, bromo, iodo, methyl, ethyl, propyl, phenyl, methoxy, ethoxy, propoxy, and phenoxy.

### Definitions

The precatalyst complexes described herein have at least one metallic center comprising a transition metal (“M”). Examples of transition metals include, but are not limited to, transition metals of Groups 9, 10 and 11 of the periodic table. Group 9 metals include Co, Rh and Ir. Group 10 elements include Ni, Pd and Pt. Group 11 elements include Cu, Ag and Au.

As used herein, the term “about” or “approximately”, when used in connection with a measurable numerical variable, refers to the indicated value of the variable and to all values of the variable that tare within the experimental error of the indicated value (e.g., within 95% confidence limit for the mean) or within ±10%, preferably ±5%, of the indicated value, whichever is greater.

As used herein, the term “Ad” refers to an adamantyl group, i.e. a tricyclo bridged hydrocarbon of formula (—C₁₀H₁₅). 1-Adamantyl may be written as (—C(CH)₃(CH₂)₆) and 2-adamantyl may be written as (—CH(CH)₄(CH₂)₅).

As used herein, the term “tBu” refers to a tert-butyl group, i.e. a branched alkyl group of formula (—C₄H₉), which may also be written as (—C(CH₃)₃).

As used herein, the term “iPr” refers to an iso-propyl group, i.e. a branched alkyl group of formula (—C₃H₇), which may also be written as (—CH(CH₃)₂).

As used herein, the term “alkyl” refers to a saturated hydrocarbon chain, such as, but not limited to, methyl, ethyl, propyl and butyl. The alkyl group may be straight-chain or branched-chain. For example, as used herein, propyl encompasses both n-propyl and iso-propyl; butyl encompasses n-butyl, sec-butyl, iso-butyl and tert-butyl, and so forth.

As used herein, the term “cycloalkyl” refers to a saturated hydrocarbon cyclic group, such as, but not limited to, cyclopropyl, cyclobutyl, cyclopentyl and cyclohexyl (Cy). Also included are bridged saturated hydrocarbon (poly) cyclic groups such as, but not limited to, adamantyl.

As used herein, the term “aryl” refers to an aromatic hydrocarbon group. Aryl includes, e.g., phenyl, biphenyl, naphthyl, anthracenyl, and so forth, as well as the substituted forms of each.

As used herein, the term “heteroaryl” refers to an aromatic group containing one or more heteroatoms. Preferably, said heteroatoms are selected from O, N and/or S. Heteroaryl includes, e.g., furyl, thienyl, pyrrolyl, pyrazolyl, imidazolyl, oxazolyl, thiazolyl, pyridyl, pyridazinyl, pyrimidinyl, pyrazinyl, 1,3,5-triazinyl, indolyl, benzofuranyl, benzoxazolyl, isoquinolyl, quinolyl, quinazolinyl, quinoxalinyl, benzoxazinyl, purinyl, pteridinyl and so forth, as well as the substituted forms of each.

“Substituted” as used herein means that one or more hydrogen atoms of the described compound or functional group is replaced with another functional group, or substituent. For example, substituted phenyl may include one or more substituents in place of any hydrogen atom on the phenyl ring. In some embodiment, there may be one substituent at the ortho, meta or para position. In other embodiments, there may be substituents at both ortho positions or both meta positions. In still other embodiments, the optionally substituted phenyl may include substituents at, e.g., both the ortho and para positions, or both meta and para positions. In some embodiments with multiple substituents, the substituents are all the same, in other embodiments with multiple substituents, the substituents are different from each other. Typical substituents include, but are not limit to, C₁-C₄ alkyl, C₁-C₄ haloalkyl and C₁-C₄ alkoxy. When a functional group is described as “optionally substituted” that functional group may have one or more substituents or no substituents.

### EXAMPLES

### Synthesis of Phosphine Compounds and Related Palladium Precatalysts

### Example 1. Synthesis of AdQPhos (1) and its Derivatives (2) and (3)

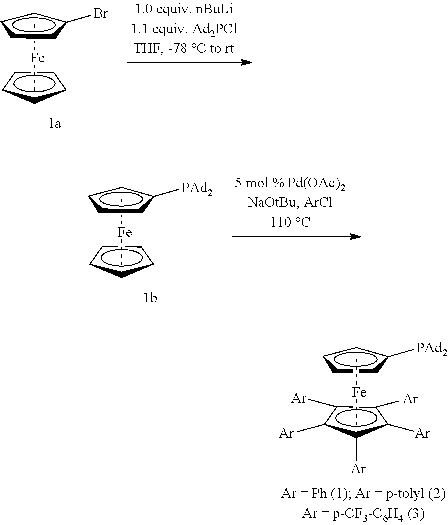

### Step 1: Synthesis of di-1-adamantylphosphinoferrocene (1b)

A 50 mL Schlenk flask containing a PTFE coated stir bar was charged with 1-bromoferrocene (1a, 4.0 g, 15.1 mmol). The container was closed with a rubber septum, evacuated, and filled with nitrogen. This cycle was repeated two additional times. Further, the dry THF (40 mL) was added, and the mixture was stirred for five minutes. The solution was cooled to −78° C., and nBuLi (2.6 M in hexanes, 5.7 mL, 15.1 mmol) was added using an additional flask for 15 minutes. The mixture was stirred for 1 h, and significant precipitation was observed (indicating the lithiation). Later, di(1-adamantyl) chloro phosphine in THF (0.75 M, 15.1 mmol) was added dropwise. The solution was allowed to warm to room temperature and stirred overnight (18 h). Considerable precipitation was observed after 18 h, and an aliquot ³¹P NMR analysis of the reaction mixture indicated complete consumption of starting material. The solution was cooled to 0-5° C. using an ice bath. The pure product was crashed-out and filtered under nitrogen. The obtained solid was washed with ether and pentane to get the pure product 1b (yellow solid, 5.6 g, 76.6%).

### Step 2: Synthesis of AdQPhos (1)

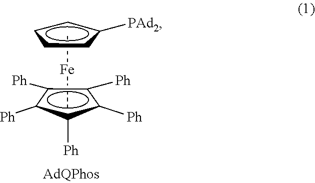

A 50 mL three-neck round bottom flask containing a PTFE coated stir bar was charged with 1b (0.972 g, 2 mmol), sodium tert-butoxide (1.9 g, 20 mmol), and palladium acetate (22.4 mg, 0.1 mmol). The container was evacuated and filled with nitrogen. This cycle was repeated two additional times. Later, chlorobenzene (20 mL, 197.2 mmol) was added to solubilize the reactants. The mixture was stirred at rt for 15 minutes. Further, the mixture was refluxed at 110-120° C. for 18 h. The reaction aliquot analysis using ³¹P NMR indicated the complete consumption of 1b. Later, the mixture was cooled to rt, diluted with DCM (20 mL), and filtered through a celite plug. The celite plug was washed with DCM to elute the remaining products. The solvents were removed under reduced pressure. The obtained dark-reddish solid was washed with acetone to yield the final product 1 (1.2 g, 71%). ¹H NMR (500 MHz, CD₂Cl₂) δ 7.33-7.31 (m, 10H), 7.14-7.07 (m, 15H), 4.68 (d, J=5 Hz, 2H), 4.47 (d, J=5 Hz, 2H), 1.92-1.89 (m, 6H), 1.58-1.57 (m, 6H), 1.58-1.57 (m, 12H), 1.50-1.47 (m, 6H); ³¹P NMR (202 MHz) 18.4 ppm.

Products (p-tolyl) AdQPhos (2) and (p-CF₃—C₆H₄) AdQPhos (3) were synthesized similarly except replacing chlorobenzene with appropriate chloroarenes.

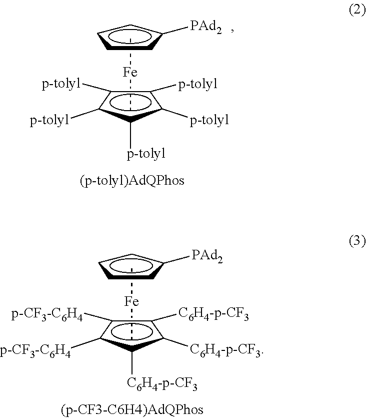

(p-tolyl) AdQPhos (2): ¹H NMR (500 MHz, CD₂Cl₂) δ7.15 (d, J=10 Hz, 10H), 6.87 (d, J=10 Hz, 10H), 4.56 (d, J=5 Hz, 2H), 4.34 (d, J=5 Hz, 2H), 2.25 (s, 15H), 1.86-1.84 (m, 6H), 1.64-1.61 (m, 6H), 1.54-1.53 (m, 12H), 1.44-1.42 (m, 6H); ³¹P NMR (202 MHz) 18.9 ppm.

(p-CF₃—C₆H₄) AdQPhos (3): ¹H NMR (500 MHz, CD₂Cl₂) δ7.43-7.39 (m, 20H), 4.69 (d, J=5 Hz, 2H), 4.49 (d, J=5 Hz, 2H), 4.88 (s, 4H), 1.85-1.82 (m, 6H), 1.64-1.52 (m, 18H), 1.44-1.41 (m, 6H); ³¹P NMR (202 MHz) 16.5 ppm.

### Example 2. Synthesis of Palladium Precatalyst (Pd-1.2) (Allyl/Crotyl/Cinnamyl Type)

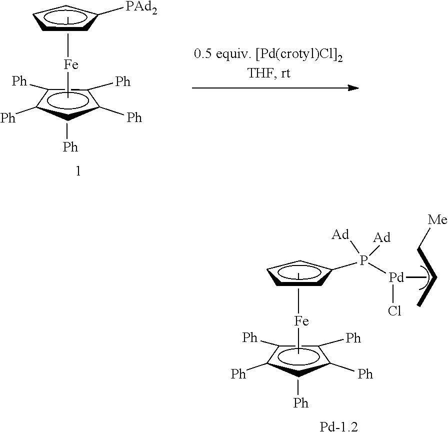

A 20 mL Schlenk flask containing a PTFE coated stir bar was charged with AdQPhos (1, 346.4 mg, 0.4 mmol) and palladium precursor (80 mg, 0.2 mmol). The container was closed with a rubber septum, evacuated, and filled with nitrogen. This cycle was repeated two additional times. Further, the dry THF (5 mL) was added, and the mixture was stirred for 2 h. Later, the ³¹P NMR analysis of the reaction aliquot indicated the complete consumption of ligand 1. The solvent was removed, and the obtained solid was washed with pentane to yield the final product Pd-1.2 (412 mg, 96%).

(AdQPhos) Pd(crotyl)Cl (Pd-1.2): ¹H NMR (500 MHz, CD₂Cl₂) δ7.26-7.23 (m, 9H), 7.19-7.12 (m, 6H), 7.10-7.03 (m, 10H), 5.14 (bs, 1H), 4.81-4.73 (m, 2H), 4.63 (bs, 2H), 4.2-4.15 (m, 1H), 3.70-3.67 (m, 1H), 2.20-2.04 (m, 10H), 1.86-1.77 (m, 9H), 1.62-1.52 (m, 15H); ³¹P NMR (202 MHz) 61.0 ppm.

### Example 3. Synthesis of Palladium Precatalyst (Pd-1.4) (Palladacycle G3 Type)

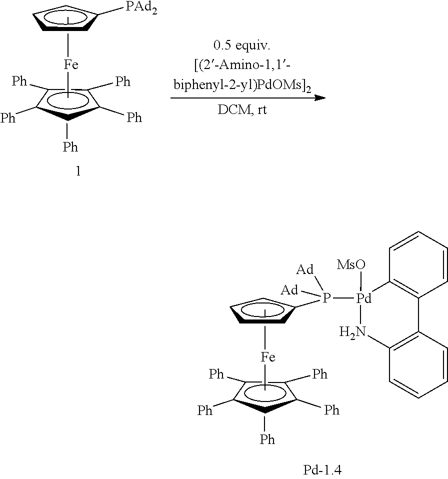

A 20 mL Schlenk flask containing a PTFE coated stir bar was charged with AdQphos (1, 346.4 mg, 0.4 mmol) and palladium precursor (148 mg, 0.2 mmol). The container was closed with a rubber septum, evacuated, and filled with nitrogen. This cycle was repeated two additional times. Further, the dry DCM (5 mL) was added, and the mixture was stirred for 2 h. Later, the ³¹P NMR analysis of the reaction aliquot indicated the complete consumption of ligand 1. The solvent was removed, and the obtained solid was washed with pentane to yield the final product Pd-1.4 (349 mg, 94%).

(AdQPhos) Pd G3 (Pd-1.4): ¹H NMR (500 MHz, CD₂Cl₂) δ7.48-7.43 (m, 1H), 7.40-7.35 (m, 1H), 7.30-7.25 (m, 2H), 7.21-7.06 (m, 27H), 6.98-6.93 (m, 1H), 6.62-6.59 (m, 1H), 4.89 (bs, 1H), 4.42 (bs, 1H), 4.15 (bs, 1H), 3.99 (bs 1H), 2.52 (bs 3H), 2.04 (bs, 6H), 1.83-1.78 (m, 9H), 1.60-1.57 (m, 12H), 1.42-1.40 (m, 3H); ³¹P NMR (202 MHz) 58.3 ppm (bs).

### Example 4. Synthesis of Palladium Precatalyst (Pd-1.7) (Palladacycle G6 Type)

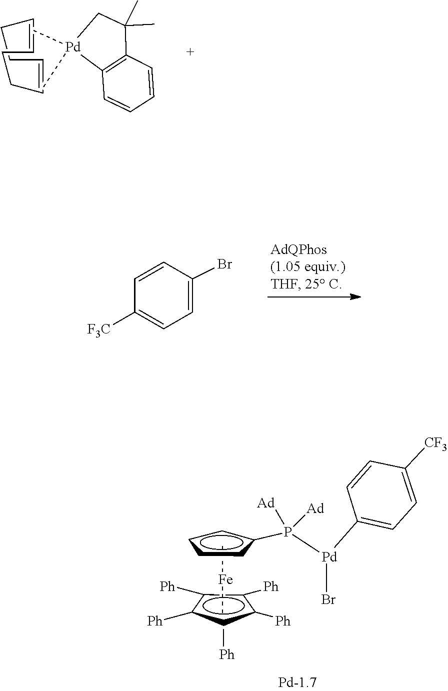

A 20 mL Schlenk flask containing a PTFE coated stir bar was charged with AdQphos (1, 182 mg, 0.21 mmol) bromoarene (67 mg, 0.3 mmol) and palladium precursor (78.1 mg, 0.2 mmol). The container was closed with a rubber septum, evacuated, and filled with nitrogen. This cycle was repeated two additional times. Further, the dry THF (5 mL) was added, and the mixture was stirred for 16 h. Later, the ³¹P NMR analysis of the reaction aliquot indicated the complete significant consumption of AdQPhos. The solvent was removed, and the obtained solid was washed with pentane to yield the final product Pd-1.7 (133 mg, 53%).

(AdQPhos) Pd G6 (Pd-1.7): ¹H NMR (500 MHz, CD₂Cl₂) δ7.42-7.07 (m, 34H), 4.80 (d, J=10 Hz, 2H), 4.61 (d, J=5 Hz, 2H), 1.93-1.87 (m, 6H), 1.78-1.74 (m, 9H), 1.60-1.54 (m, 15H); ³¹P NMR (202 MHz) 41.2 ppm(s).

### Example 5. Synthesis of AdMPhos (4) and its Precatalyst (Pd-4.1)

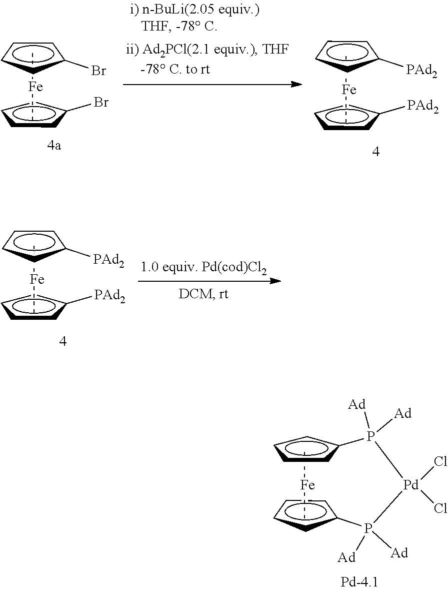

A 50 mL Schlenk flask containing a PTFE coated stir bar was charged with 1-bromoferrocene (4a, 1.0 g, 3.77 mmol). The container was closed with a rubber septum, evacuated, and filled with nitrogen. This cycle was repeated two additional times. Further, the dry THF (20 mL) was added, and the mixture was stirred for five minutes. The solution was cooled to −78° C., and nBuLi (2.6 M in hexanes, 2.9 mL, 7.72 mmol) was added using an additional flask for 15 minutes. The mixture was stirred for 2 h, and significant precipitation was observed (indicating the lithiation). Later, di(1-adamantyl) chloro phosphine in THF (0.75 M, 8.0 mmol) was added dropwise. The solution was allowed to warm to room temperature and stirred overnight (18 h). Considerable precipitation was observed after 18 h, and an aliquot ³¹P NMR analysis of the reaction mixture indicated considerable consumption of starting material. The solution was cooled to 0-5° C. using an ice bath. The pure product was crashed-out and filtered under nitrogen. The obtained solid was washed with ether and pentane to get the pure product 4 (yellow solid, 0.72 g, 62%).

A 20 mL Schlenk flask containing a PTFE coated stir bar was charged with AdMPhos (4, 100.0 mg, 0.13 mmol) and palladium precursor (36.3 mg, 0.13 mmol). The container was closed with a rubber septum, evacuated, and filled with nitrogen. This cycle was repeated two additional times. Further, the dry DCM (5 mL) was added, and the mixture was stirred for 5 h. Later, the ³¹P NMR analysis of the reaction aliquot indicated the complete consumption of ligand 4. The solvent was removed, and the obtained solid was washed with pentane to yield the final product Pd-4.1 (113 mg, 90%).

(AdMPhos)PdCl₂ (Pd-4.1): ¹H NMR (500 MHz, cd₂cl₂) δ4.71-4.28 (m, 8H), 2.44-1.44 (m, 60H); ³¹P NMR (202 MHz) 59.6 ppm.

### Application of Palladium Precatalysts in Palladium-Catalyzed Organic Transformations

### Example 6. Palladium-Catalyzed sp²-sp³ Coupling Reaction

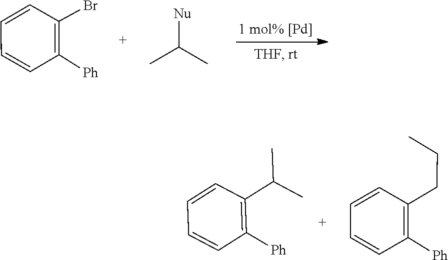

A 20 mL vial was charged with various precatalysts (1.0 mol %), 2-bromobiphenyl (0.17 mL, 1.0 mmol, 1.0 equiv.) and a stirring bar. The mixture was dissolved in THF (5 mL). Then, an isopropyl-Nu solution (iPrZnBr: 4.0 mL, 0.5 M, 2.0 mmol, 2.0 equiv.) was added dropwise and stirred for 6 h at rt. The reaction yields were determined by GC. For the reaction with isopropyl lithium, the Ferringa's procedure was used. The individual reactions are shown in Table 1 below.

TABLE 1
Reactions of Example 6

| | Nucleophile | | |
|---|---|---|---|
| Entry | [Nu] | Precatalyst [Pd] | GC Yield (%)⁽*⁾ |
| 1 | Li | AdQPhos (1):Pd₂(dba)₃ | 99 (32.3:1) |
| 2 | ZnBr | AdQPhos (1):Pd₂(dba)₃ | 29.9 (3:1) |
| 3 | ZnBr | QPhos:Pd₂(dba)₃ | traces |
| 4 | ZnBr | AdQPhosPd(crotyl)Cl | 84 (2.9:1) |
| | | (Pd-1.2) | |
| 5 | ZnBr | AdQPhosPdG3 (Pd-1.4) | 89 (3:1) |
| 6 | ZnBr | AdMPhosPdCl₂ (Pd-4.1) | 75 (4.2:1) |

⁽*⁾GC Yield:ratio refers to 2-isopropylbiphenyl:2-(n-propyl)biphenyl.

2-Isopropylbiphenyl: ¹H NMR (500 MHz, CDCl₃) δ7.48-7.43 (m, 3H), 7.41 (d, J=10 Hz, 2H), 7.36-7.33 (m, 2H), 7.28-7.22 (m, 2H), 3.18-3.15 (m, 1H), 1.21 (d, J=10 Hz, 6H) ppm.

2-Isopropylnaphthanene: ¹H NMR (500 MHz, CDCl₃) δ7.82-7.78 (m, 3H), 7.65 (s, 1H), 7.5-7.39 (m, 3H), 3.08-3.15 (m, 1H), 1.35 (d, J=10 Hz, 6H) ppm.

### Example 7. Palladium-Catalyzed C—O Coupling Reaction

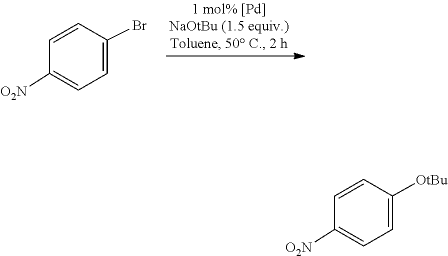 [SMILES: O=[N+]([O-])c1ccc(Br)cc1 ; CC(C)(C)Oc1ccc([N+](=O)[O-])cc1]

A 4 mL vial was charged with various precatalysts (1.0 mol %), 4-nitrobromobenzene (0.17 mL, 1.0 mmol, 1.0 equiv.), sodium tert-butoxide (60 mg, 0.62 mmol, 1.25 equiv.) and a stirring bar. The mixture was dissolved in toluene (2 mL). The mixture was then stirred at 50° C. Reaction yields were determined by GC. The individual reactions are shown in Table 2 below.

TABLE 2
Reactions of Example 7

| Entry | Precatalyst [Pd] | GC Yield (%) |
|---|---|---|
| 1 | AdQPhos (1):Pd₂(dba)₃ | 99% (isolated yield 89%) |
| 2 | (p-tolyl)AdQPhos (2):Pd₂(dba)₃ | 90% |
| 3 | (p-CF₃-C₆H₄)AdQPhos (3):Pd₂(dba)₃ | 91% |
| 4 | QPhos:Pd₂(dba)₃ | 74% |

4-Tert-butoxy-nitrobenzene: ¹H NMR (500 MHz, CDCl₃) δ8.16 (d, J=10 Hz, 2H), 7.04 (d, J=5 Hz, 2H), 1.46 (s, 9H) ppm.

### Example 8. Palladium-Catalyzed C—S Coupling Reaction

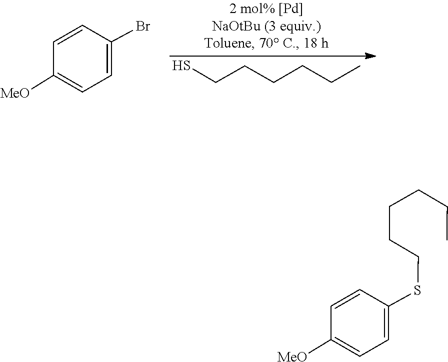

A 4 mL vial was charged with various precatalysts (2.0 mol %), 4-bromoanisole (0.063 mL, 0.5 mmol, 2.0 equiv.), sodium tert-butoxide (72 mg, 0.75 mmol, 3.0 equiv.) and a stirring bar. The mixture was dissolved in toluene (2 mL). Later, hexane thiol (0.035 mL, 0.25 mmol, 1.0 equiv.) was added and the mixture was then stirred at 70° C. Reaction yields were determined by GC. The individual reactions are shown in Table 3 below.

TABLE 3
Reactions of Example 8

| Entry | Precatalyst [Pd] | GC Yield (%) |
|---|---|---|
| 1 | AdQPhos (1):Pd₂(dba)₃ | 86% (isolated yield 63%) |
| 2 | AdQPhosPd(crotyl)Cl (Pd-1.2) | 44 |
| 3 | AdMPhosPdCl₂ (Pd-4.1) | 60 |

1-(Hexylthio)-4-methoxybenzene: ¹H NMR (500 MHz, CDCl₃) δ7.33 (d, J=10 Hz, 2H), 6.84 (d, J=10 Hz, 2H), 3.85 (s, 3H), 2.81 (t, J=10 Hz, 2H), 1.60-1.55 (m, 2H), 1.46-1.55 (m, 2H), 1.30-1.26 (m, 4H), 0.88 (t, J=10 Hz, 3H) ppm.

### Example 9: Palladium-Catalyzed α-Arylation of Ketones

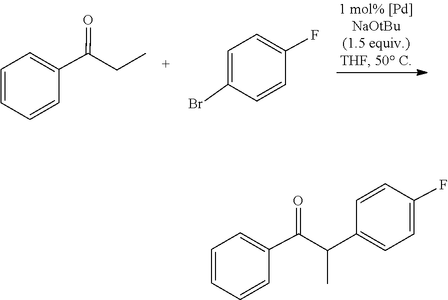

A 20 mL vial was charged with various precatalysts (1.0 mol %), 4-fluorobromobenzene (0.26 mL, 2.0 mmol, 2.1 equiv.), ethyl phenyl ketone (0.14 mL, 1.0 mmol, 1.0 equiv.), sodium tert-butoxide (153 mg, 1.5 mmol, 1.5 equiv.) and a stirring bar. The mixture was dissolved in THF (5 mL). The mixture was then stirred at 50° C. Reaction yields were determined by GC. The individual reactions are shown in Table 4 below.

TABLE 4
Reactions of Example 9

| Entry | Precatalyst [Pd] | GC Yield (%) |
|---|---|---|
| 1 | AdQPhosPd(crotyl)Cl (Pd-1.2) | 84 |
| 2 | AdQPhosPdG3 (Pd-1.4) | 75 |
| 3 | AdQPhos (1):Pd₂(dba)₃ | 99 |
| 4 | QPhos:Pd₂(dba)₃ | 98 |
| 5 | AdMPhosPdCl₂ (Pd-4.1) | 89 |

### Product:

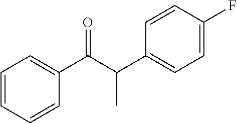 [SMILES: CC(C(=O)c1ccccc1)c1ccc(F)cc1]

¹H NMR (500 MHz, CDCl₃) δ7.96 (d, J=10 Hz, 2H), 7.42 (d, J=10 Hz, 2H), 7.29-7.26 (m, 2H), 7.02-6.99 (m, 2H), 4.71 (q, J=10 Hz, 1H)_{1.54} (d, J=5 Hz, 3H) ppm.

### Example 10: Palladium-Catalyzed α-Arylation of Amides

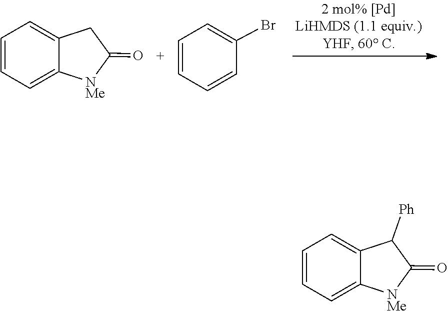

A 20 mL vial was charged amide (73.59 mg; 0.50 mmol; 1.00 eq.), precatalyst (2 mol %) and a stirring bar. The mixture was dissolved in THF Then, bromobenzene (0.06 ml; 0.55 mmol; 1.10 eq.) was added and it was stirred for 5 minutes. Then, lithium bis(trimethylsilyl)amide solution, 1.0 M in THF (0.55 ml; 0.55 mmol; 1.10 eq.) was added in portion and the mixture was stirred at 70° C. for 20 h. Conversion was measured using GC. The individual reactions are shown in Table 5 below.

TABLE 5
Reactions of Example 10

| Entry | Precatalyst [Pd] | GC Yield (%) |
|---|---|---|
| 1 | AdQPhos (1):Pd₂(dba)₃ | 44 |
| 2 | (p-tolyl)AdQPhos (2):Pd₂(dba)₃ | 20 |
| 3 | QPhos:Pd₂(dba)₃ | traces |
| 4 | AdQPhosPd(crotyl)Cl (Pd-1.2) | 79 |
| 5 | AdQPhosPdG3 (Pd-1.4) | 83 (74% isolated) |

### Product:

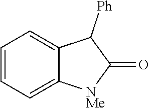 [SMILES: CN1C(=O)C(c2ccccc2)c2ccccc21]

¹H NMR (500 MHz, CDCl₃) δ7.37-7.33 (m, 3H), 7.30-7.28 (m, 1H), 7.21-7.16 (m, 3H), 7.06 (t, J=10 Hz, 1H), 6.90 (d, J=10 Hz, 1H), 4.61 (s, 1H), 3.26 (s, 3H) ppm.

### Example 11: Palladium-Catalyzed α-Arylation of Nitroalkanes

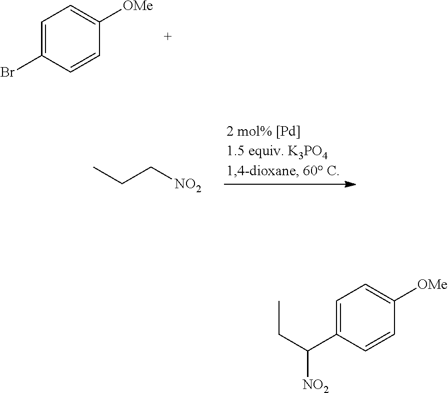

A 20 mL vial was charged with precatalyst (2 mol %), nitropropane (0.45 mL; 5.0 mmol; 10 eq.), 4-bromoanisole (0.06 mL, 0.5 mmol, 1.0 equiv.), K₃PO₄ (127.2 mg, 0.75 mmol, 1.5 equiv.), and stirring bar. The mixture was dissolved in 1,4-dioxane. Later the mixture was stirred at 60° C. for 20 h. Conversion was measured using GC. The individual reactions are shown in Table 6 below.

TABLE 6
Reactions of Example 11

| Entry | Precatalyst [Pd] | GC Yield (%) |
|---|---|---|
| 1 | AdQPhos (1):Pd₂(dba)₃ | 39 |
| 2 | (p-tolyl)AdQPhos (2):Pd₂(dba)₃ | 53 |
| 3 | QPhos:Pd₂(dba)₃ | traces |
| 4 | AdQPhosPd(crotyl)Cl (Pd-1.2) | 89 (80% isolated) |
| 5 | AdQPhosPdG3 (Pd-1.4) | 84 |

### Example 12: Palladium-Catalyzed α-Arylation of Nitriles

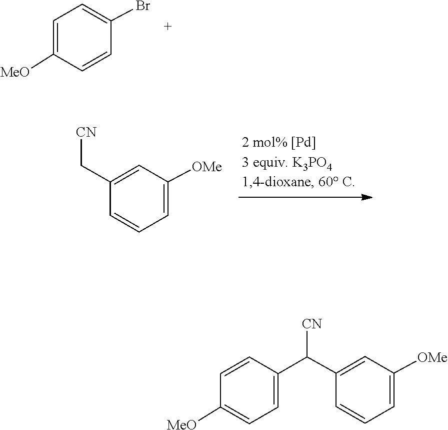 [SMILES: COc1ccc(Br)cc1 ; COc1cccc(CC#N)c1 ; COc1ccc(C(C#N)c2cccc(OC)c2)cc1]

A 20 mL vial was charged with precatalyst (2 mol %), nitrile (0.07 mL; 0.5 mmol; 1.0 eq.), 4-bromoanisole (0.06 mL, 0.5 mmol, 1.0 equiv.), K₃PO₄ (318 mg, 1.5 mmol, 3.0 equiv.), and a stirring bar. The mixture was dissolved in 1,4-dioxane. Later the mixture was stirred at 60° C. for 20 h. Conversion was measured using GC. The individual reactions are shown in Table 7 below.

TABLE 7
Reactions of Example 12

| Entry | Precatalyst [Pd] | GC Yield (%) |
|---|---|---|
| 1 | AdQPhos (1):Pd₂(dba)₃ | 11 |
| 2 | (p-tolyl)AdQPhos (2):Pd₂(dba)₃ | 27 |
| 3 | QPhos:Pd₂(dba)₃ | traces |
| 4 | AdQPhosPd(crotyl)Cl (Pd-1.2) | 73 |
| 5 | AdQPhosPdG3 (Pd-1.4) | 60 |

### Example 13: Palladium-Catalyzed α-Arylation of Esters

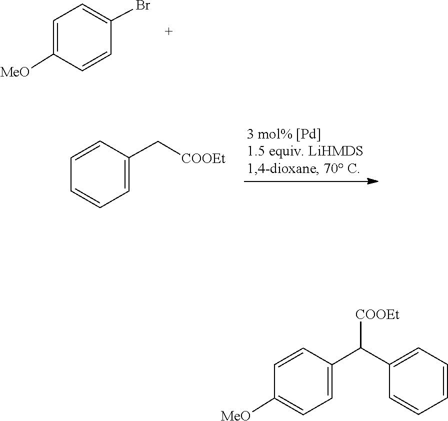 [SMILES: COc1ccc(Br)cc1 ; CCOC(=O)Cc1ccccc1 ; CCOC(=O)C(c1ccccc1)c1ccc(OC)cc1]

A 20 mL vial was charged with precatalyst (3 mol %), ester (200 mg, 1.22 mmol, 2.4 equiv.) and a PTFE coated stirring bar. Later, dry toluene was added and the mixture was stirred for 2 minutes. LiHMDS (1.25 mmol) was added to this solution at 0° C. This mixture was stirred for 10 minutes. Later, the solution of 4-bromoanisole in in 0.5 mL toluene was added drop wise. The mixture was then stirred at 70° C. for 20 h. Conversion was measured using GC. The individual reactions are shown in Table 8 below.

TABLE 8
Reactions of Example 13

| Entry | Precatalyst [Pd] | GC Yield (%) |
|---|---|---|
| 1 | AdQPhos (1):Pd₂(dba)₃ | 89 |
| 2 | (p-tolyl)AdQPhos (2):Pd₂(dba)₃ | 64 |
| 3 | (p-CF₃-C₆H₄)AdQPhos (3):Pd₂(dba)₃ | 50 |
| 4 | QPhos:Pd₂(dba)₃ | traces |
| 5 | AdQPhosPd(crotyl)Cl (Pd-1.2) | 99 |
| 6 | AdQPhosPdG3 (Pd-1.4) | 60 |
| 7 | AdMPhosPdCl₂ (Pd-4.1) | 73 |

### Example 14: Palladium-Catalyzed P—C Coupling Reaction

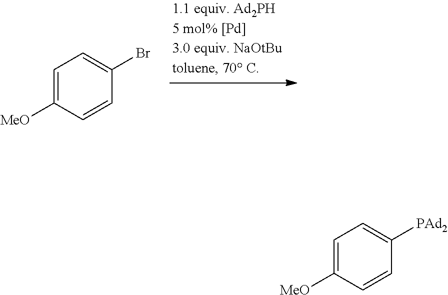

A 4 mL vial was charged with various precatalysts (5.0 mol %), 4-bromoanisole (0.032 mL, 0.25 mmol, 1.0 equiv.), sodium tert-butoxide (72 mg, 0.75 mmol, 3.0 equiv.) and a stirring bar. The mixture was dissolved in toluene (2 mL). Later, Ad₂PH (83 mg, 0.25 mmol, 1.1 equiv.) was added and the mixture was then stirred at 70° C. Reaction yields were determined by ³¹P NMR. The individual reactions are shown in Table 9 below.

TABLE 9
Reactions of Example 14

| Entry | Precatalyst [Pd] | Yield Product (%) |
|---|---|---|
| 1 | AdQPhos (1):Pd₂(dba)₃ | 88.5 |
| 2 | AdQPhosPd(crotyl)Cl (Pd-1.2) | 92.5 |
| 3 | AdMPhosPdCl₂ (Pd-4.1) | 33 |
| 4 | QPhos:Pd₂(dba)₃ | 80 |

The examples provided herein are by no way intended to limit the scope of the invention as set forth in the claims.

### REFERENCES

(1) (a) D. S. Surry, S. L. Buchwald, Chem. Sci. 2011, 2, 27-50; (b) C. C. C. Johansson Seechurn, M. O. Kitching, T. J. Colacot, V. Snieckus, Angew. Chem. Int. Ed. 2012, 51, 5062-5085.
(2) (a) G. A. Grasa, T. J. Colacot, Org. Lett. 2007, 9, 5489-5492; (b) R. C. J. Atkinson, N. J. Long, Monodentate Ferrocene Donor Ligands, in P. Stepnicka, Ferrocenes: Ligands, Materials and Biomolecules, Wiley, 2008; (c) T. J. Colacot, Chem. Rev. 2003, 103, 3101-3118.
(3) G. P. Sollott, H. E. Mertwoy, S. Portnoy, J. L. Snead, J. Org. Chem. 1963, 28, 1090-1092.
(4) G. R. Knox, P. L. Pauson, D. Willison, Organometallics 1992, 11, 2930-2933.
(5) S. Juge, J. P. Genet, Tetrahedron Lett. 1989, 30, 2783-2786.
(6) E. A. Colby, T. F. Jamison, J. Org. Chem. 2003, 68, 156-166.
(7) N. Kataoka, Q. Shelby, J. P. Stambuli, J. F. Hartwig, J. Org. Chem. 2002, 67, 5553-5566.
(8) (a) N. A. Strotman, H. R. Chobanian, J. He, Y. Guo, P. G. Dormer, C. M. Jones, J. E. Steves, J. Org. Chem. 2010, 75, 1733-1739; (b) D. Cheng, J. Liu, D. Han, G. Zhang, W. Gao, M. H. Hsieh, N. Ng, S. Kasibhatla, C. Tompkins, J. Li, et al., ACS Med. Chem. Lett. 2016, 7, 676-680; (c) Y. Ohtake, T. Sato, T. Kobayashi, M. Nishimoto, N. Taka, K. Takano, K. Yamamoto, M. Ohmori, M. Yamaguchi, K. Takami, et al., J. Med. Chem. 2012, 55, 7828-7840.
(9) C. C. C. Johansson Seechurn, S. L. Parisel, T. J. Colacot, J. Org. Chem. 2011, 76, 7918-7932.

1. A compound of Formula I: 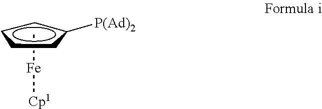
wherein:
Cp¹ is represented by Formula II or Formula III 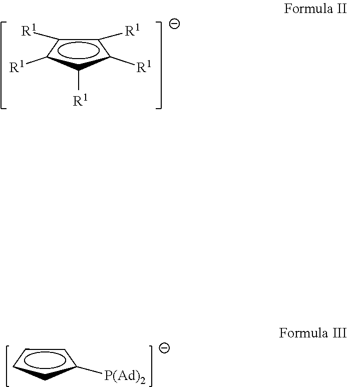
R¹ is at each occurrence independently from each other selected from optionally substituted C₆-C₁₀ aryl or C₃-C₉ heteroaryl;
Ad is adamantyl; and
each optional substituent, when present, is selected from the group consisting of C₁-C₄ alkyl, C₁-C₄ haloalkyl, and C₁-C₄ alkoxy.

2. The compound according to claim 1, wherein the compound is represented by Formula IV 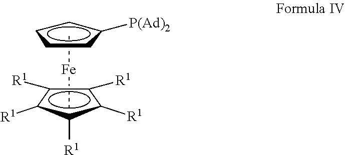
wherein R¹ and Ad are defined as in claim 1.

3. The compound according to claim 1, wherein R¹ is at each occurrence independently from each other selected from optionally substituted C₆ aryl; and each optional substituent, when present, is selected from the group consisting of C₁-C₄ alkyl, C₁-C₄ haloalkyl, and C₁-C₄ alkoxy.

4. The compound according to claim 1, wherein the compound is represented by Formula V: 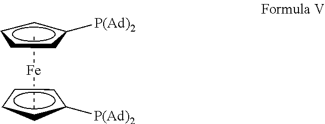
wherein Ad is defined as in claim 1.

5. The compound according to claim 1, wherein the compound is selected from the group consisting of

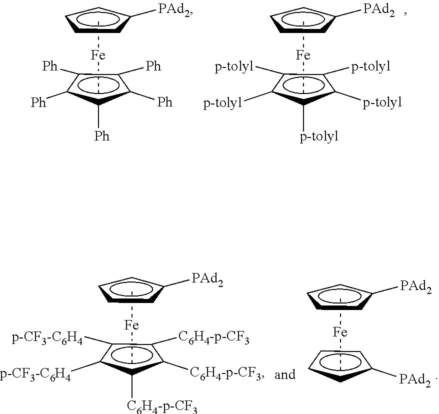

6. A process for synthesizing a compound according to claim 2 comprising the following steps:
(a) lithiation of a compound according to Formula A and reacting it with Ad₂PY² to obtain a compound according to Formula B; and
(b) reacting the compound according to Formula B with R¹Y³ in the presence of a base and a catalytic amount of Pd(OAc)₂ to obtain a compound according to Formula IV; 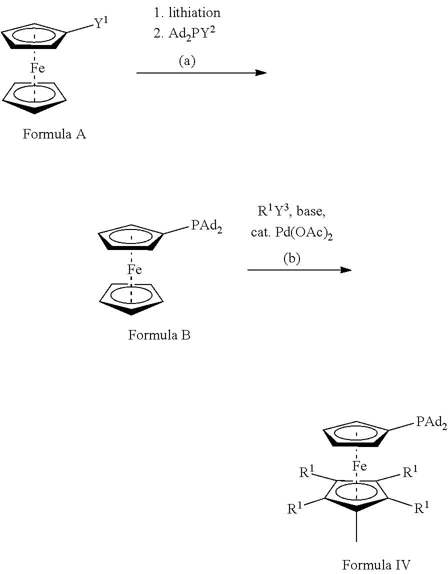
wherein:
Y¹ is selected from a halogen atom or a hydrogen atom;
Y², Y³ are independently from each other selected from a halogen atom;
R¹ is at each occurrence independently from each other selected from optionally substituted C₆-C₁₀ aryl or C₃-C₉ heteroaryl;
Ad is adamantyl; and
each optional substituent, when present, is selected from the group consisting of C₁-C₄ alkyl, C₁-C₄ haloalkyl, and C₁-C₄ alkoxy.

7. The process according to claim 6, wherein the lithiation in step (a) is carried out by reacting the compound according to Formula A with an organolithium reagent.

8. The process according to claim 6, wherein the base in step (b) is an alkali metal alkoxide or an alkaline earth metal alkoxide.

9. A process for synthesizing a compound according to claim 4 comprising the following step:
(a′) lithiation of a compound according to Formula C and reacting it with Ad₂PY² to obtain a compound according to Formula V: 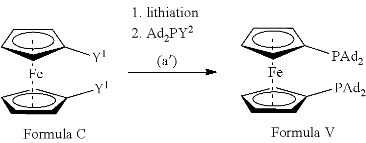
wherein:
Y¹ is selected from a halogen atom or a hydrogen atom;
Y² is selected from a halogen atom; and
Ad is adamantyl.

10. The process according to claim 9, wherein the lithiation in step (a′) is carried out by reacting the compound according to Formula C with an organolithium reagent.

11. A precatalyst of Formula VI or Formula VII: 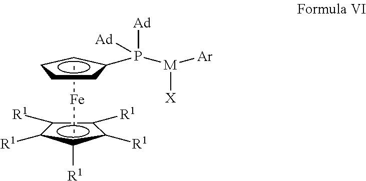
wherein:
R¹ is at each occurrence independently from each other selected from optionally substituted C₆-C₁₀ aryl or C₃-C₉ heteroaryl;
Ad is adamantyl;
M is a transition metal selected from Group 9, Group 10 or Group 11;
X is selected from the group consisting of halide, triflate (TfO⁻), tetrafluoroborate (BF₄⁻), hexafluorophosphate (PF₆⁻), mesylate (MsO⁻), tosylate (TsO⁻), tetrakis[3,5-bis(trifluoromethyl)phenyl]borate (B[(CF₃)₂C₆H₃]₄⁻), hexafluoroantimonate (SbF₆⁻) and combinations thereof;
Ar is optionally substituted C₆-C₁₀ aryl; and
each optional substituent, when present, is selected from the group consisting of C₁-C₄ alkyl, C₁-C₄ haloalkyl, and C₁-C₄ alkoxy.

12. The precatalyst according to claim 11, wherein R¹ is at each occurrence independently from each other selected from optionally substituted C₆ aryl; and each optional substituent, when present, is selected from the group consisting of C₁-C₄ alkyl, C₁-C₄ haloalkyl, and C₁-C₄ alkoxy.

13. The precatalyst according to claim 11, wherein M is selected from the group consisting of Co, Rh, Ir, Ni, Pd, Pt, Cu, Ag, and Au.

14. The precatalyst according to claim 11, wherein Ar is optionally substituted C₆ aryl.

15. The precatalyst according to claim 11, wherein the precatalyst is selected from the group consisting of 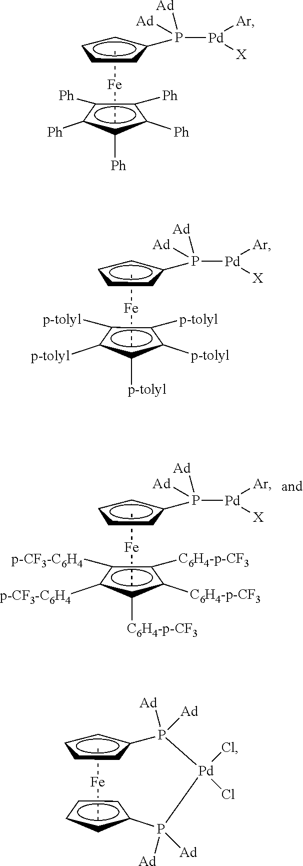
wherein:
Ad is 1-adamantyl;
Ar is Ph or p-(trifluoromethyl)phenyl; and
X is selected from the group consisting of chloride (Cl⁻), bromide (Br⁻), triflate (TfO⁻) and mesylate (MsO⁻).

16. A precatalyst of Formula VIII or Formula IX: 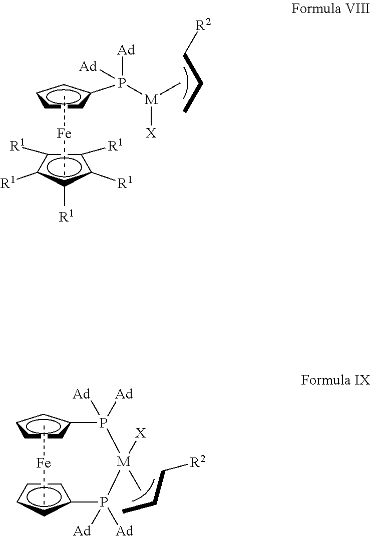
wherein:
R¹ is at each occurrence independently from each other selected from optionally substituted C₆-C₁₀ aryl or C₃-C₉ heteroaryl;
Ad is adamantyl;
M is a transition metal selected from Group 9, Group 10 or Group 11;
X is selected from the group consisting of halide, triflate (TfO⁻), tetrafluoroborate (BF₄⁻), hexafluorophosphate (PF₆⁻), mesylate (MsO⁻), tosylate (TsO⁻), tetrakis[3,5-bis(trifluoromethyl)phenyl]borate (B[(CF₃)₂C₆H₃]₄⁻), hexafluoroantimonate (SbF₆⁻) and combinations thereof;
R² is selected from the group consisting of H, C₁-C₄ alkyl and C₆-C₁₀ aryl; and
each optional substituent, when present, is selected from the group consisting of C₁-C₄ alkyl, C₁-C₄ haloalkyl, and C₁-C₄ alkoxy.

17. The precatalyst according to claim 16, wherein R¹ is at each occurrence independently from each other selected from optionally substituted C₆ aryl; and each optional substituent, when present, is selected from the group consisting of C₁-C₄ alkyl, C₁-C₄ haloalkyl, and C₁-C₄ alkoxy.

18. The precatalyst according to claim 16, wherein M is selected from the group consisting of Co, Rh, Ir, Ni, Pd, Pt, Cu, Ag, and Au.

19. The precatalyst according to claim 16, wherein the precatalyst is selected from the group consisting of 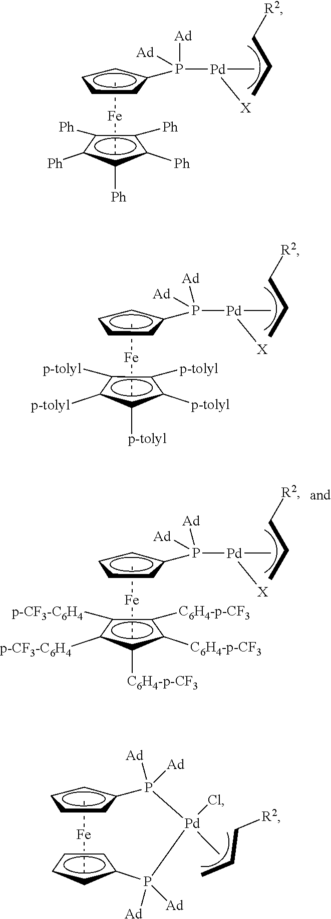
wherein:
Ad is 1-adamantyl;
R² is selected from the group consisting of H, Me, and Ph; and
X is selected from the group consisting of chloride (Cl⁻), bromide (Br⁻), iodide (I⁻), triflate (TfO⁻), tetrafluoroborate (BF₄⁻) and hexafluorophosphate (PF₆⁻).

20. A precatalyst of Formula X or Formula XI: 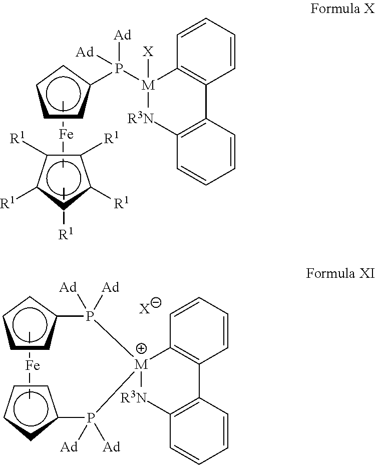
wherein:
R¹ is at each occurrence independently from each other selected from optionally substituted C₆-C₁₀ aryl or C₃-C₉ heteroaryl;
Ad is adamantyl;
M is a transition metal selected from Group 9, Group 10 or Group 11;
X is selected from the group consisting of halide, triflate (TfO⁻), tetrafluoroborate (BF₄⁻), hexafluorophosphate (PF₆⁻), mesylate (MsO⁻), tosylate (TsO⁻), tetrakis[3,5-bis(trifluoromethyl)phenyl]borate (B[(CF₃)₂C₆H₃]₄⁻), hexafluoroantimonate (SbF₆⁻) and combinations thereof;
R³ is selected from the group consisting of H, Me, NHMe, Ph, and NHPh; and
each optional substituent, when present, is selected from the group consisting of C₁-C₄ alkyl, C₁-C₄ haloalkyl, and C₁-C₄ alkoxy.

21. The precatalyst according to claim 20, wherein R¹ is at each occurrence independently from each other selected from optionally substituted C₆ aryl; and each optional substituent, when present, is selected from the group consisting of C₁-C₄ alkyl, C₁-C₄ haloalkyl, and C₁-C₄ alkoxy.

22. The precatalyst according to claim 20, wherein M is selected from the group consisting of Co, Rh, Ir, Ni, Pd, Pt, Cu, Ag, and Au.

23. The precatalyst according to claim 20, wherein the precatalyst is selected from the group consisting of 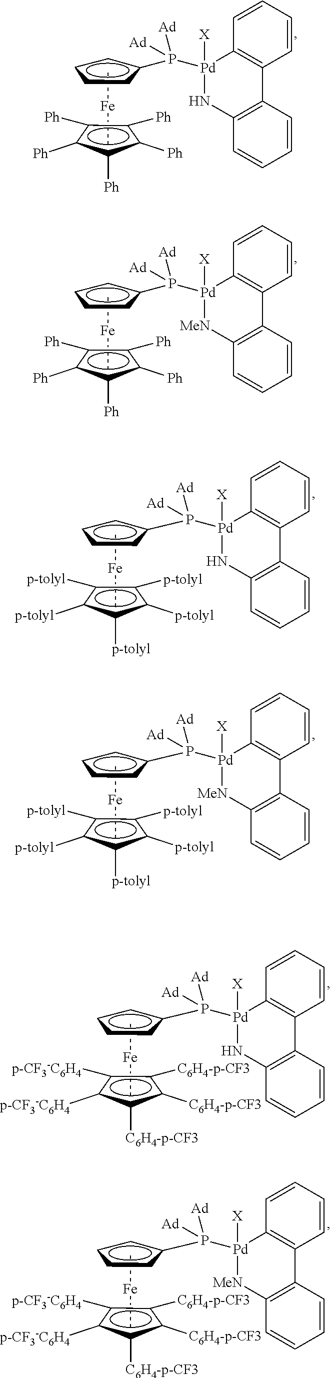 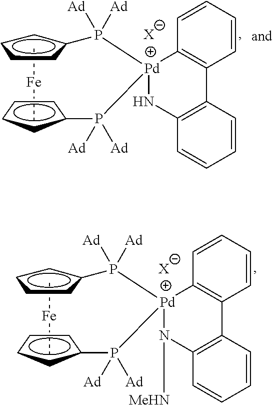
wherein:
Ad is 1-adamantyl; and
X is mesylate (MsO⁻).

24. A method for performing a transition metal-catalyzed coupling reaction between a first substrate and a second substrate, wherein the method comprises the following steps:
(a) providing a compound according to claim 1 and a transition metal source in a reaction vessel;
(b) adding a first substrate and a second substrate to the reaction vessel; and
(c) reacting the first substrate and the second substrate at a temperature and a time sufficient to perform a transition metal-catalyzed coupling reaction.

25. The method according to claim 24, wherein the transition metal source is a Pd metal source.

26. The method according to claim 24, wherein the transition metal-catalyzed coupling reaction is selected from a C(sp²)-C(sp²) coupling reaction, a C(sp²)-C(sp³) coupling reaction, a C(sp²)-N coupling reaction, a C(sp²)-O coupling reaction, a C(sp²)-S coupling reaction, a C(sp²)-P coupling reaction or an α-arylation of an amide, ester, nitrile, nitroalkane or ketone.

27. A method for performing a transition metal-catalyzed coupling reaction between a first substrate and a second substrate, wherein the method comprises the following steps:
(a) providing a precatalyst according to claim 11 in a reaction vessel;
(b) adding a first substrate and a second substrate to the reaction vessel; and
(c) reacting the first substrate and the second substrate at a temperature and a time sufficient to perform a transition metal-catalyzed coupling reaction.

28. The method according to claim 27, wherein the precatalyst is a Pd precatalyst.

29. The method according to claim 27, wherein the transition metal-catalyzed coupling reaction is selected from a C(sp²)-C(sp²) coupling reaction, a C(sp²)-C(sp³) coupling reaction, a C(sp²)-N coupling reaction, a C(sp²)-O coupling reaction, a C(sp²)-S coupling reaction, a C(sp²)-P coupling reaction or an α-arylation of an amide, ester, nitrile, nitroalkane or ketone.
